(19) United States
(12) Patent Application Publication
(10) Pub. No.: US 2005/0037386 A1
(43) Pub. Date: Feb. 17, 2005

(54) Composition and processes for analysis of pharmacologic agents in biological samples

(75) Inventors: Janet F. Morrison, Simsbury, CT (US); John S. Sundsmo, Vista, CA (US); Carl M. Selavka, Charlton, CT (US); David H. Brill, Schuylerville, NY (US)

Correspondence Address: FITZPATRICK CELLA HARPER & SCINTO, 30 ROCKEFELLER PLAZA, NEW YORK, NY, 10112, US

(73) Assignee: DRUG RISK SOLUTIONS, L.L.C.

(21) Appl. No.: 10/840,016

(22) Filed: May 5, 2004

Related U.S. Application Data
(60) Provisional application No. 60/467,894, filed on May 5, 2003.

(51) Int. Cl. C12Q 1/68; G01N 33/53; G01N 33/48

(52) U.S. Cl. 435/6; 435/7.1; 436/63

(57) ABSTRACT

A method for extracting and detecting analytes that are metabolically associated with biological samples, as well as, for distinguishing between those which are environmentally associated and those which are metabolically incorporated is disclosed.

### CROSS REFERENCE TO RELATED APPLICATIONS

This application claims priority under 35 U.S.C. § 119(e) to U.S. Provisional Application No. 60/467,894, filed May 5, 2003.

### BACKGROUND OF THE INVENTION

1. Field of the Invention

The invention relates generally to compositions and processes for analyzing biological samples for chemical agents and specifically to methods of using supercritical fluids to simultaneously extract analytes from biological samples.

2. Related Art

Increased drug abuse in North America has been associated with criminal activities, health problems, newborn addiction, lost worker productivity and staggeringly high medical costs. Currently of greatest concern are opiates (heroin, morphine, codeine), cocaine, marijuana, amphetamine, methamphetamine, MDMA (Ecstasy), and phencyclidine.

Possible pesticide residues in the breast tissues of women (Stellman, S. D.; Djordjevic, M. V.; Muscat, J. E.; Gong, L.; Bernstein, D.; Citron, M. L.; White, A.; Kemeny, M.; Busch, E.; Nafziger, A. N. 2000. Relative abundance of organochlorine pesticides and polychlorinated biphenyls in adipose tissue and serum of women in Long Island, N.Y. Cancer Epidemiology, Biomarkers and Prevention. 9: 1241-1249; have raised concerns about possible environmental exposure including air- and water-borne agents; of additional concern is the exposure of domestic animals to agricultural chemical agents such as pesticides and herbicides. Verifying a natural “organic” status prior to slaughter has, thus, recently become of significant interest.

In testing for human drugs of abuse, several test systems are presently marketed for detecting drug analytes in urine e.g., ONTRAK™ and ONLINE™ (Roche Diagnostic Systems, Inc.), the ADx™ automated fluorescence polarization immunoassay system (Abbott Laboratories, Inc.) and EZ-SCREEN™ (Environmental Diagnostics). Data relating to certain cross-reactivity and performance testing results have been disclosed (Schilling et al., 1999; Smith et al., 1997). Unfortunately, there are significant problems associated with urine testing for drugs of abuse, e.g., (i) possible false positive results for opiates recorded in subjects who are on certain medications and who have recently ingested poppy seeds; (ii) rapid elimination rates and short half-life of many drug metabolite compounds; and particularly (iii) false negatives associated with purposeful sample adulteration and interference (Mikkelsen et al., 1988).

Unlike liquid urine samples, solid samples such as hair require special sample preparation prior to conducting assays. Baumgartner and co-workers (Baumgartner, A. M., Jones, P. F., Baumgartner, W. A., Black, C. T. 1979. J. Nucl. Med. 20: 748-752) reported in 1979 the use of chemical and enzymatic methods to isolate drug analytes from human hair samples for subsequent radioimmunoassay. Detection of opiates was disclosed in extracts of hair from suspected heroin abusers. Conceptually, hair provides a better toxicological specimen than urine, serum, sweat or saliva because its relatively slow growth may increase the period of time during which drug usage is detectable. In present day practice, a variety of wet-chemical extraction methods are employed to release “matrix-bound” drugs from hair, including acid incubation and/or base hydrolysis procedures, prolonged enzymatic digestion, organic solvent extraction and/or sonication. These labor-intensive methods require technical experience and are presently most easily conducted in a test laboratory. However, even then the assays frequently suffer from poor reproducibility, long delays before results can be released and variability in the ability to isolate different drugs and their metabolites. Hydrolysis conditions can also result in conversion of drug metabolites such as 6-monoacetylmorphine, i.e., whose presence provides judicial proof of drug abuse, into parent compounds, i.e., morphine. Fortunately, it has been found that certain drugs and their metabolites can persist in hair for extended periods of time, as evidenced by detection of cocaine and its metabolites in the hair of South American Indians (Henderson, G. L., Harkey, M. R., Zhou, C., Jones, R. T. 1992. J. Anal. Toxicol. 16: 199-201) and Bolivian mine workers (Moller, M. R., Fey, P., Rimbach, S. 1992. J. Anal. Toxicol. 16: 291-296) identified as daily chewers of coca leaves.

In a legal setting, the probative advantages of hair testing may often depend critically on methods used to assess how a drug analyte might have become associated with hair, i.e., discrimination between the possibility of environmental contamination (i.e., through passive exposure) and the possibility of metabolic incorporation. Pre-extraction steps commonly used to address the issue of environmental contamination include at least: (i) extensive tedious rinsing procedures designed to theoretically remove a drug that is “passively associated” with hair and (ii) use of kinetic measurements to assess how rapidly a drug is released from hair samples under different conditions. Commercial test laboratories engaged in hair drug testing disclose a wide variety of operationally-defined rinsing protocols to remove environmental contamination from the hair surface; however, the significance and effectiveness of these decontamination procedures have been the subject of much debate (Kidwell, D. A.; Blank, D. L. In Drug Testing in Hair, Kintz, P., ed.; CRC Press, Inc.: Boca Raton, Fla., 1996, Chapter 2; Wang, W. L.; Cone, E. J. Forensic Sci. Int. 1995, 70, 39-51; Blank, D. L.; Kidwell, D. A. Forensic Sci. Int. 1993, 63, 145-156; Baumgartner, W. A.; Hill, V. A. Forensic Sci. Int. 1993, 63, 157-160; Blank, D. L.; Kidwell, D. A. Forensic Sci. Int. 1995, 70, 13-38). Observed individual sample variations, possibly related to methodology, have raised doubts about the ability of hair testing to discriminate between environmental contamination and metabolic incorporation, i.e., the issues have included possibility of differences based in hair porosity, type of hair, amount of environmental drug exposure, hair chemical treatments and the like. Possible methods for discriminating between environmental exposure and metabolic incorporation have included kinetic and mathematical assessments of concentrations of analytes in wash fluids, e.g. assessments of a “curvature ratio” and an “extended wash ratio”; and a hair porosity testing method utilizing methylene blue (e.g., Baumgartner et al., 1996). As an additional issue, variability has been reported in the ability to isolate metabolically incorporated analytes from certain hair samples, i.e., even after rinsing, pulverization, grinding, cutting and/or various wet chemical extraction methods. It has been assumed that the nature of different hair matrices may vary, necessitating certain individualized treatments. While individualized treatment may be feasible with limited numbers of samples in a research laboratory setting, there are significant problems with such an approach in a commercial setting. Clearly, it would be highly valuable to identify the chemical basis of drug association with hair so that conditions may be optimized for the variety of different types as may be encountered in commercial testing.

Based on findings that weakly basic drugs associated well with hair (e.g., cocaine), and that weakly acidic drugs associated poorly (e.g., 11-nor-delta-9-tetrahydrocannabinol-9-carboxylic acid), Kidwell and Blank (Kidwell, D. A.; Blank, D. L. In Drug Testing in Hair, Kintz, P., ed.; CRC Press, Inc.: Boca Raton, Fla., 1996, Chapter 2) suggested predominant interactions of drug cationic residues with protein anionic side chain residues in acidic amino acids (e.g. Asp and Glu). Cone and Joseph (Cone, E. J.; Josephs, R. D. 1996. In: Drug Testing in Hair. Ed. Kintz, P. CRC Press, Boca Raton, Fla.) suggested a possible association of certain drugs with melanin hair pigment. Morrison et al. (Morrison, J. F.; MacCrehan, W. A., 1994. Proceedings of the 5th International Symposium on Supercritical Fluid Chromatography and Extraction, Baltimore, Md., Jan. 11-14, 1994, Supercritical Conferences: Cincinnati, Ohio, , p. F-16; Morrison, J. F.; Chesler, S. N.; Yoo, W. J.; Selavka, C. M. . 1998. Anal. Chem. 70, 163-167) studied possible mechanisms for association of cocaine and benzoylecgonine (BZE) with hair using CO₂ as a fluid under supercritical conditions of temperature and pressure. Large differences were reported in recovery of cocaine from artificially-fortified hair and drug user hair. Extractability of cocaine, but not BZE, was reportedly increased in the presence of triethylamine (TEA) and water, suggesting to the authors (Morrison, J. F.; Chesler, S. N.; Yoo, W. J.; Selavka, C. M. . 1998. Anal. Chem. 70, 163-167) a possible cationic interaction of drug with hair and an ion exchange mechanism wherein TEA displaced cocaine at the hair binding site. Limited availability of confirmed drug user hair samples has occasioned the use of hair which has been “fortified” with drugs by prolonged incubation in solutions of DMSO containing the target drug analytes. Differences have been reported between the extractability of drugs from drug-fortified and drug-user hair (Morrison, J. F.; Chesler, S. N.; Yoo, W. J.; Selavka, C. M. 1998. Anal. Chem. 70, 163-167).

Supercritical fluids, comprising gases held at pressures and temperature above their critical points, possess physical properties intermediate between liquids and gases offering possibilities of increased diffusivity, solubility and extactability. Carbon dioxide is desirable because its critical pressure and temperature are relatively low, i.e., P_{c}=73 atm and T_{c}=31° C., respectively, and it is relatively non-toxic and non-polluting. Supercritical CO₂ (SF-CO₂) has been used for supercritical fluid extraction (SFE), supercritical fluid chromatography (SFC) and enhanced fluidity liquid chromatography (pcSFC). The low intrinsic polarity of CO₂ creates special challenges in solubilizing polar compounds. Brewer et al. (Brewer, W. E.; Galipo, R. C.; Sellers, K. W.; Morgan, S. L. 2001. Analysis of cocaine, benzoylecgonine, codeine, and morphine in hair by supercritical fluid extraction with carbon dioxide modified with methanol. Anal. Chem. 73: 2371-2376) disclosed that supercritical extraction of cocaine from drug-fortified hair using SF-CO₂ was slow, giving incomplete removal and poor recoveries in the range of 35%. Sachs and co-workers (Sachs, H. and Raff, I., Comparison of quantitative results of drugs in human hair by GC/MS, Forensic Sci. Int., 63, 207, 1993) disclosed SFE of opiates and cocaine from drug-fortified hair using CO₂ modified with ethyl acetate at 300 atm and 60° C. Edder et al. (Edder, P.; Staub, C.; Veuthey, J. L.; Pierroz, I.; Haerdi, W. 1994 J. Chromatogr. B, 658, 75-86) disclosed recovery of opiates, i.e., codeine, morphine, ethylmorphine and monoacetylmorphine, from drug-fortified and drug-user hair using subcritical CO₂, i.e., 40° C., 25 MPa, modified with a ternary mixture of methanol, triethylamine, and water (85:6:6:3% v/v). Veuthey et al. (Veuthey, J. L.; Edder, P.; Staub, C. 1995. Analysis, 23, 258-265) disclosed possible SFE isolation of cocaine, benzoylecgonine, methadone, codeine, morphine, ethylmorphine and monoacetylmorphine from drug-fortified hair with CO₂/methanol/triethylamine/water (98:0.8:0.8:0.4 v/v). Morrison and co-workers (Morrison, J. F.; MacCrehan, W. A., 1994. Proceedings of the 5th International Symposium on Supercritical Fluid Chromatography and Extraction, Baltimore, Md., Jan. 11-14, 1994, Supercritical Conferences: Cincinnati, Ohio, p. F-16; Morrison, J. F.; Chesler, S. N.; Yoo, W. J.; Selavka, C. M. 1998. Anal. Chem. 70, 163-167) disclosed SFE recoveries of cocaine from drug-fortified and drug-user hair using CO₂ at 110° C. and 400 atm following addition of a modifier mixture of water and triethylamine directly to the hair sample . However, the recovery of the metabolite benzoylecgonine (BZE) was reportedly only about 11% that attained by acid hydrolysis, suggesting inability to desorb BZE under these conditions. Cirimele et al.( Cirimele, V.; Kintz, P.; Majdalani, R.; Mangin, P. 1995. J. Chromatogr. B., 673, 173-181) disclosed recovery from drug-user hair of opiates, i.e., codeine, morphine, and 6-monoacetylmorphine, cocaine, delta-9-tetrahydrocannabinol, cannabidiol and cannabinol using CO₂ modified with methanol-triethylamine-water (2:2:1 v/v) under supercritical conditions of 100° C. and 315 bar. Brewer and colleagues (Brewer, W. E.; Galipo, R. C.; Sellers, K. W.; Morgan, S. L. 2001. Analysis of cocaine, benzoylecgonine, codeine, and morphine in hair by supercritical fluid extraction with carbon dioxide modified with methanol. Anal. Chem. 73: 2371-2376) suggested a possible method for SFE isolation of cocaine, benzoylecgonine, codeine, and morphine from hair using supercritical CO₂ modified with 10% methanol at 300 atm and 145° C. Plaut et al. (Plaut, O.; Girod, C.; Staub, C. 1998. Forensic Sci. Int., 92, 219-227) disclosed isolation of methaqualone from hair using SFE with CO₂/methanol/water (85:12:3 v/v) at 350 atm and 60° C. Allen and Oliver (Allen, D. L.; Oliver, J. S. 2000. The use of supercritical fluid extraction for the determination of amphetamines in hair. Forensic Sci.Int. 107: 191-197) disclosed recoveries of methylenedioxyamphetamine (MDA), methylenedioxymethamphetamine (MDMA), and methylenedioxyethylamphetamine (MDEA) from spiked and drug user hair by SFE using CO₂/chloroform/isopropanol (90:9:1 v/v) at 3800 psi and 70° C. Morrison and Rada (Morrison, J. F.; Rada, A. L. 2000. Abstracts of the 220th National Meeting of the American Chemical Society, Paper No. 412229, Washington, D.C., August 24) disclosed a possible SFE with in situ derivatization of amphetamine, methamphetamine, and MDMA from drug-fortified and spiked hair using CO₂ modified with ethyl acetate and heptafluorobutyric acid anhydride (HFBA). Brewer et al. (Brewer, W. E.; Galipo, R. C.; Sellers, K. W.; Morgan, S. L. 2001. Analysis of cocaine, benzoylecgonine, codeine, and morphine in hair by supercritical fluid extraction with carbon dioxide modified with methanol. Anal. Chem. 73: 2371-2376) disclosed low SFE recoveries of cocaine (in the range of 36%) from fortified hair using chloroform as a modifier at 380 atm and 50° C., causing these authors to conduct further work focusing on just methanol as modifier. Unfortunately, continuous flow of modifier was required over about 70 minutes at 300 atm and 145° C. to achieve optimal isolation of cocaine from fortified hair. An optimal commercial method for extraction of chemical analytes from hair would desirably consist of a just a few steps, and preferably, just a single set of extraction conditions capable of yielding high recoveries for all analytes of interest in less than one hour.

Once isolated, the presence or amount of a drug analyte in a hair sample may, in concept, be determined using certain immunoassay formats and/or physico-chemical analytical methods such as gas chromatography (GC), liquid chromatography (LC), mass spectrometry (MS), tandem GC/MS and/or LC/MS, and the like. Extracts, however, often contain interfering background materials (e.g., endogenous co-extractable components). Similarly, extracts and hair digests often contain interfering, cross-reacting and background-enhancing substances in standardized immunoassay protocols. Thus, it is often necessary to conduct a preliminary analysis on a crude extract or digest using a first test system (i.e., referred to as a screening assay), and if a positive result is obtained, to conduct additional confirmatory testing (i.e., confirmation testing). Confirmation testing procedures typically involve isolation of the target drug analytes from the crude extracts by liquid-liquid extraction (LLE) and/or solid-phase extraction (SPE) prior to quantification by dedicated test equipment, e.g., GC/MS, GC/tandem-MS, GC/ion-trap MS, or LC/MS.

Thus, there is a need for improved hair test extraction methodologies and assays, particularly those which might be used in relatively unsophisticated testing environments where highly trained laboratory personnel are not commonly available. Objects of the invention solve variability problems in the extraction of drug and chemical analytes from hair samples and provide rapid, and efficient alternatives to existing commercial hair extraction methods and assays.

### SUMMARY OF THE INVENTION

Methods are disclosed for simultaneously and/or tandemly extracting and detecting drug and chemical analytes that are associated with biological samples such as hair, feathers, nails, hoofs, skin and muscle. The methods utilize supercritical fluid extraction (SFE) in CO₂ containing optional modifiers and/or additives. The methods achieve simultaneous and/or tandem-extraction of polar and non-polar drug analytes, or, alternatively, derivatizatized analogs of polar drug analytes. Derivatization of the drug analytes stabilizes chemical structure, improves extraction and offers benefits of increased speed, ease of extraction, opportunities for immediate GC/MS analysis, reduced cost, faster throughput, simultaneous closed-loop on-line extraction and detection, as well as opportunities for automation. Modifiers and additives optimize conditions for rapid release of analytes from hair glycoproteins and complex carbohydrates. Uses include determining exposure of humans and domestic animals to certain drugs of abuse, illegal agricultural drug substances, pesticides, herbicides, environmental toxins and carcinogens.

In one embodiment of the invention, a method for determining the presence of an analyte in a sample collected from a subject comprising simultaneous on-line extraction of an analyte from the sample into a supercritical fluid and isolation of a derivatized analyte therefrom and determination of the amount of derivatized analyte in the hair sample.

In another embodiment of the invention, there is provided a supercritical fluid extractor method for screening a subject to determine the presence of a drug analyte comprising extracting any analyte present in a biological sample from the subject by treating the sample with a derivatizing agent to neutralize the charge of the analyte and extracting the analyze from the sample into a supercritical fluid and testing whether any drug is present in the fluid.

The invention further provides a method for supercritical fluid extraction of an analyte having a reactive hydrogen group from a biological sample, comprising treating the sample with a derivatizing agent prior to initiating supercritical fluid extraction.

In yet a further embodiment of the invention, there is provided a method for supercritical fluid simultaneous extraction of two or more analytes from a biological sample, wherein the analytes have a reactive hydrogen group and a reactive carboxyl group comprising treating the sample with a derivatizing agent effective to derivatize both the inactive hydrogen and the reactive carboxyl.

The invention further comprises a method for testing a biological sample to determine whether it is exposed to analyte in the environment or whether to analyte in the environment or whether the analyte is metabolically incorporated in the sample, comprising placing the sample in an equipment pack having a modifier and an additive in an extraction chamber that can operate in static and dynamic flow mode of a supercritical fluid, initiating a dynamic flow mode effective in washing the sample to remove analyte environmentally associated with the sample but not analyte metabolically incorporated into the sample, collecting the wash for testing at a later time, then initiating a static mode to extract metabolically associated analyte with the modifier and optional additive in the equipment pack, switching again to dynamic mode to extract and collect the metabolically associated extract for testing at a later time, testing the wash sample and extracted sample to determine whether they include analyte wherein a positive signal in the wash sample and not in extract sample suggests that the analyte is environmentally associated with the sample and wherein a positive in the extract suggests the analyte is metabolically associated with the sample.

Further provided is a method for extracting analyte from a hair sample from a subject comprising incubating the sample with an enzyme that can degrade and hydrolyze the hair matrix glycoconjugate. This may also be accomplished by treating the sample with a derivatizing agent effective to neutralize the chemical charge of one or more sugar ring substituent groups in the matrix and extracting the analytes in a supercritical fluid.

### BRIEF DESCRIPTION OF THE DRAWINGS

FIG. 1 depicts the steps in a chemical reaction mechanism wherein acyl addition at reactive hydrogen atoms in amphetamine results in both improved extractability and simultaneous derivatization in a supercritical CO₂ fluid.

FIG. 2 is a digital tracing depicting both a gas chromatographic separation (upper panel) and mass spectrometric analysis (lower panel) of the molecular derivative ions produced following simultaneous extraction and acyl derivatization of methamphetimine and amphetamine drug analytes in a supercritical CO₂ fluid.

FIG. 3 depicts a chemical reaction mechanism for esterification of carboxylic acid groups in a drug analyte (R′(O)OH) with an acid anhydride results in a pentafluoropropyl-ester-drug analyte-derivative with improved extractability in a supercritical CO₂ fluid.

FIG. 4 illustrates the supercritical fluid extraction results (blue bars) obtained for the simultaneous isolation of (underivatized) amphetamine, methamphetamine, cocaine, PCP, and cocaethylene from a NIST hair standard reference material (NIST SRM 2379, National Institute of Standards and Technology, Gaithersburg, Md.). SFE Conditions: CO₂+methanol/water/triethylamine (2:1:2); batch addition of 900 μL of the modifier/additive mixture to the extraction vessel prior to initiation of the static SFE step. Extraction pressure=450 atm; extraction temperature=100° C.; static extraction time=15 min; dynamic extraction elution volume=30 mL CO₂; fluid flow rate=2 mL/min; nominal hair sample mass=20 mg. SFE performed on an ISCO Model SFX 3560 with extract collection into methanol. Analytes were quantified by GC-MS using the internal standard method following post-extraction derivatization with heptafluorobutyric anhydride (HFBA). Internal standards consisted of deuterated analogs of the target drug analytes. NIST-certified results are shown for comparison (red bars).

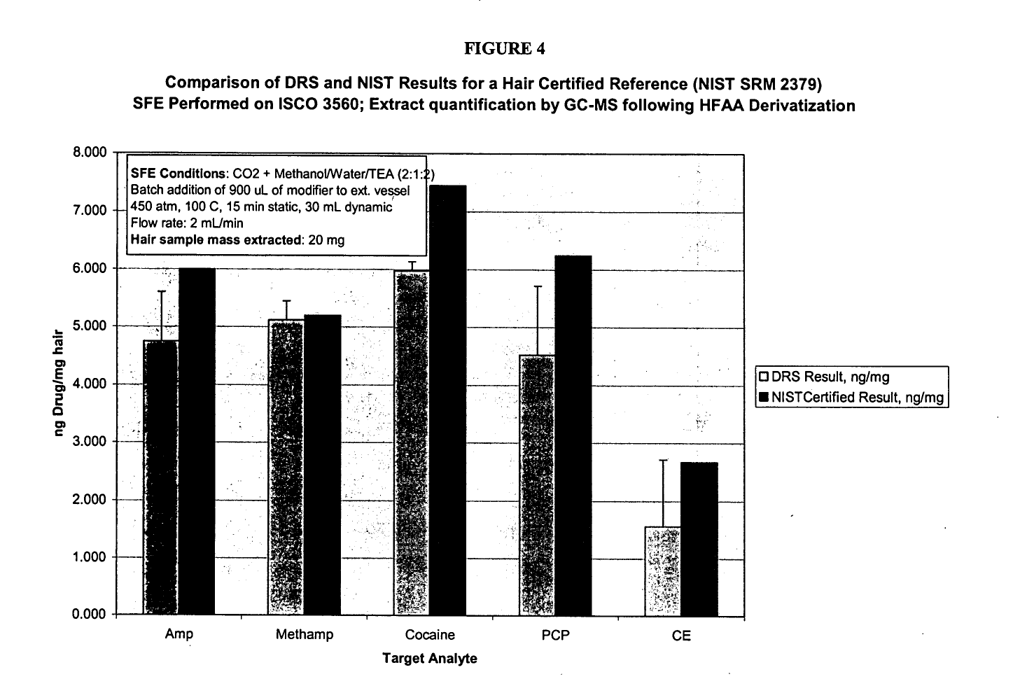

FIG. 5 illustrates the supercritical fluid extraction results (blue bars) obtained for the simultaneous isolation of (underivatized) codeine, morphine, 6-acetylmorphine, and THC from a NIST hair standard reference material (NIST SRM 2380, National Institute of Standards and Technology, Gaithersburg, Md.). Extraction and analysis conditions as in FIG. 4.

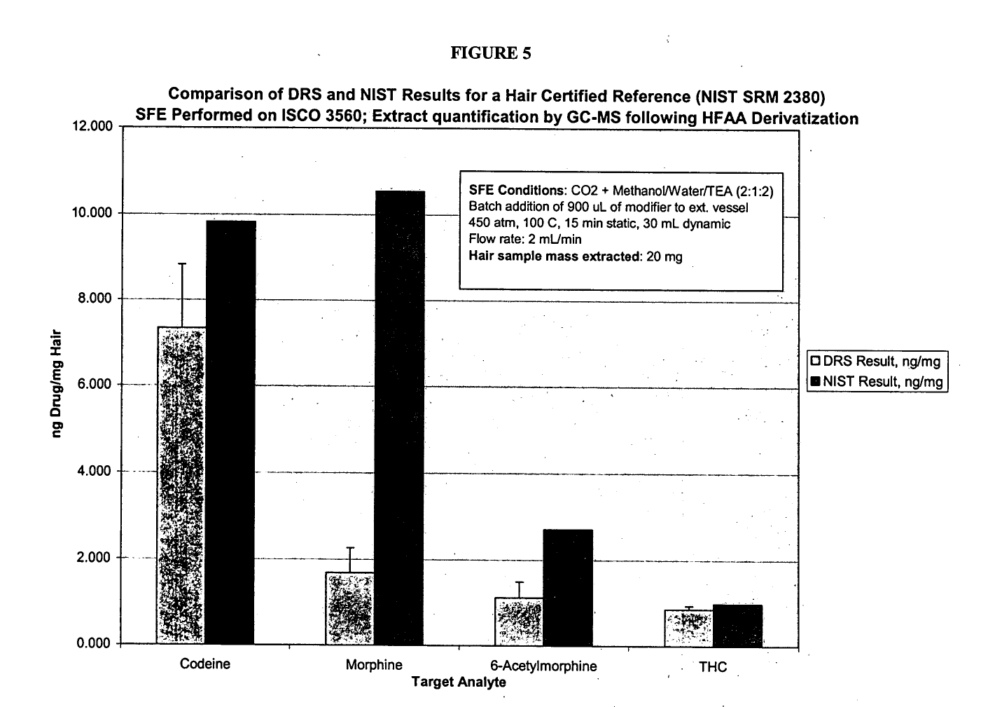

### DETAILED DESCRIPTION OF THE INVENTION

While it may have been appreciated by those in the field of forensic science that extraction of certain chemical analytes from biological samples such as hair, feather, nails, hoofs, skin and muscle, may be accomplished under certain conditions with supercritical fluid extraction, extraction of polar analytes from samples has proven problematic. Particularly, while it has been appreciated in the art that different SFE conditions may be required for isolation of chemical analytes having different chemistries, e.g., polar vs. non-polar analytes or cationic vs. anionic analytes, it is not believed that the conditions so-useful are presently understood. While many studies have utilized spiked—(drug added to hair surface) and fortified—(soaked in drug) biological samples, results with actual drug user hair have suggested different incorporation chemistry.

Hair biochemistry is perhaps at the heart of the issue. While it has been understood that hair is composed of keratin proteins, extraction of drugs of abuse from hair does not appear to follow simple rules of protein chemistry. For instance Morrison et al (Morrison, J. F.; MacCrehan, W. A., 1994. Proceedings of the 5th International Symposium on Supercritical Fluid Chromatography and Extraction, Baltimore, Md., Jan. 11-14, 1994, Supercritical Conferences: Cincinnati, Ohio, p. F-16; Morrison, J. F.; Chesler, S. N.; Yoo, W. J.; Selavka, C. M. 1998. Anal. Chem. 70, 163-167) found cocaine to be extractable by SFE using CO₂ modified with water and triethylamine, but under these conditions benzoylecgonine was inexplicably not released. Similarly, Kauert et al. (Kauert, G.; Meyer, L. V.; Herrle, I. 1992. Zbl.Rechtsmed. 28: 33) reported methanolic extraction required sonication in methanol for 5 hrs., i.e., a relatively prolonged period for solubilizing organic compounds in organic solvents. Kintz (Kintz, P. 1996. In: Drug Testing in Hair, CRC Press, Boca Raton, Fla.) reported acid hydrolysis in HCl with an incubation time of 18 hrs., again a relatively prolonged period of time for protein hydrolysis. Baumgartner et al. (U.S. Pat. Nos. 6,022,693 and 6,350,582, herein incorporated by reference in their entireties) reported overnight incubation in papain with additional possible treatments being necessary. Moeller and Fey (Moeller,M. R.; Fey, P. 1992. J. Anal. Toxicol. 16: 291) reported incubation in β-glucuronidase and arylsulfatase to be partially effective with certain analytes. Overall, even after considering the extensively cross-linked nature of keratin molecules in hair, these extraction properties are now believed by the inventors not to be strongly convincing of protein side-chain interactions being controlling in drug substance binding in hair. Re-investigating the possible physical forces at play in drug-hair interactions, particular note was taken of reported blood group substances in the medullary region of certain mammalian hair shafts (e.g., HairGsl).

Complex carbohydrates constituting the A,B,O blood group system are synthesized and expressed in mammalian cells in glycosphingolipids and protein glycoconjugates. It was reasoned that in addition to medullary blood group substances, hair shafts might contain trapped residues of other cell membrane components, e.g., phospholipids, glyceryl-fatty acids, phosphatidyl choline, phosphatidyl ethanolamine, phosphatidyl inositol, cholesterol and the like; cell membrane glycoprotein receptors; as well as, glycosphingolipids and blood group glycoconjugates. Multiple types of hair-drug physical interactions were considered possible. For example, glycolipids are amphiphilic and composed of a long-chain sphingosine base, a fatty acyl side chain (ceramide) and one or more carbohydrate residues classed e.g. as neutral lipids, and charged sialic acid containing gangliosides, sulfolipids and the like. It therefore became of considerable interest to reinvestigate how these multiple possible hair components might jointly contribute to hair-drug interactions; hydrophobic, ionic and van der Waals binding forces; as evidenced in SFE behavior.

Recognizing the value of targeting glycoconjugates during SFE, conditions were sought under which blood group substance complex carbohydrates, i.e., glycolipids and glycoproteins, associated with hair might be purposefully effected, e.g. through use of detergents and fragmention, to potentiate extraction of drugs of abuse without destruction of those drugs or their metabolites.

Options which presented themselves for consideration included “simultaneous extraction methodologies” and “tandem extraction methodologies”, as defined below.

In considering the possible solutions to the problems in the art, the inventors recognized the following: namely,

1. The theoretically favorable mass transport properties of SFs (e.g., “gas-like” low viscosities and high diffusivities) might impart them with excellent hair-matrix penetrating power, obviating the need for pulverization of the hair sample or dissolution of the keratin matrix and permitting more rapid, efficient extraction compared with liquid extractants;

2. The tunable solvation properties of SFs might impart them with selectivity advantages which would allow more effective discrimination between environmentally associated—and metabolically incorporated—drug analytes;

3. Since densities (and, hence, solvating power) of SFs approach those of liquids, this solvating power might be varied in desirable ways by changing extraction temperature, pressure, or fluid composition;

4. The number of variable parameters available with SF extraction might provide means for both optimization and individualized variations, i.e., designed to allow both overall high recoveries from the majority of samples but also selective “tuning” of the SFE conditions to individual samples, i.e., such as is not presently possible using common liquid-liquid extraction (LLE) or solid phase extraction (SPE) methods;

5. SFE might be amenable to automation, offering for the first time the potential for significant reductions in operator time and opportunity for operator error;

6. Automated SFE assay might also offer improvements in sample throughput and assay specificity, sensitivity and precision;

7. Because SFs are commonly gases at ambient conditions, post-extraction concentration steps might be greatly simplified;

8. The relatively low critical temperatures of SFs makes them particularly suitable as possible extractants for thermally labile compounds, i.e., a particular concern with certain drugs and metabolites; and,

9. SFE was also viewed as attractive from both a cost- and environmental-standpoint. Carbon dioxide is currently widely used for SFE, i.e., because of its relatively easy to attain critical parameters (31° C. and 73 atm), low toxicity and chemical inertness, low cost, and availability. Because CO₂ is a gas at ambient conditions, the generation of hazardous solvent waste might be virtually eliminated, thus eliminating and/or dramatically reducing costs and strict regulatory burdens associated with current disposal of hazardous chemical wastes in test laboratories using liquid solvents for sample preparation and extraction.

The prospects of improved extraction efficiency and selectivity, increased sample throughput, decreased sample handling and pre-treatment, decreased method development and operator time, lower solvent consumption, minimal hazardous waste generation, and simplification of post-extraction concentration steps suggested to the inventors the possible desirability of this technology for sample preparation and extraction in high throughput toxicological applications such as (on-site) drug screening.

Upon evaluation, limitations noted by the inventors in the art of SFE included the following: namely,

b 1. Sachs and co-workers (Sachs, H. and Raff, I., Comparison of quantitative results of drugs in human hair by GC/MS, Forensic Sci. Int., 63, 207, 1993) reported application of supercritical fluids to the isolation of opiates and cocaine from hair using CO₂ (at 300 atm and 60° C.) modified with ethyl acetate. GC-MS analysis of the resultant extracts demonstrated that extraction rates and reproducibility of the SFE technique under these conditions were not comparable to wet chemical methods. These investigators seemingly attempted to increase the solvent strength of the SF extraction fluid by incorporating a modifier, i.e., 20 μL of ethyl acetate added to the 150-μL extraction vessel. While ethyl acetate is known to be a solvent for the latter drugs in the liquid state, this approach seemingly failed to consider the possible influence of strong analyte-matrix binding interactions on analyte extractability, as well as differences between wet chemical fluid phase extractability and SFE;

2. Edder et al. (Edder, P.; Staub, C.; Veuthey, J. L.; Pierroz, I.; Haerdi, W. 1994 J Chromatogr. B, 658, 75-86) reported recovery of opiates from drug-fortified and drug user hair using subcritical CO₂, i.e., 40° C., 25 MPa, modified with a ternary mixture of methanol, triethylamine, and water (85:6:6:3% v/v). In these studies, water appeared necessary, but not sufficient, to achieve efficient recoveries of the target analytes from the hair matrix. GC-MS analysis of SF extracts obtained from fortified hair (prepared by soaking drugs into the hair) suggested detection in the concentration range of 0.5-2 ng/mg for the four opiates tested, i.e., levels within the range of detection of GC/MS but challenging to attain in a routine commercial immunoassay format. Replicate analyses of blank hair established a limit of GC/MS quantitation at about 0.1 ng/mg for codeine, morphine, and ethylmorphine, and 0.2 ng/mg for 6-monoacetylmorphine. As evaluated by the inventors, the reported SFE data appeared to perform better than both acidic and basic hydrolysis methods. However, while the apparent quantitative efficiency and reproducibility of this reported methodology also seemed to compare favorably with both methanol extraction and enzymatic digestion procedures followed by solid phase extraction, the recoveries of morphine were reportedly higher than those observed with the other methods while levels of 6-monoacylmorphine (6-MAM) were slightly lower. The latter findings suggested to the inventors the possibility of hydrolysis of 6-MAM to morphine during SFE under these conditions of use.

3. Morrison and co-workers (Morrison, J. F.; MacCrehan, W. A., 1994. Proceedings of the 5th International Symposium on Supercritical Fluid Chromatography and Extraction, Baltimore, Md., Jan. 11- 14, 1994, Supercritical Conferences: Cincinnati, Ohio, p. F-16; Morrison, J. F.; Chesler, S. N.; Yoo, W. J.; Selavka, C. M. . 1998. Anal. Chem. 70, 163-167) suggested in earlier disclosed work the SFE recoveries of cocaine from drug-fortified and drug user hair. In these original studies, Morrison and co-workers (Morrison, J. F.; MacCrehan, W. A., 1994. Proceedings of the 5th International Symposium on Supercritical Fluid Chromatography and Extraction, Baltimore, Md., Jan. 11-14, 1994, Supercritical Conferences: Cincinnati, Ohio, p. F-16; Morrison, J. F.; Chesler, S. N.; Yoo, W. J.; Selavka, C. M. 1998. Anal. Chem. 70, 163-167) noted that the SF extractability of cocaine and benzoylecgonine was found to be highly dependent upon the nature of the matrix and apparently upon the putative manner in which the target analytes are incorporated into or onto the matrix. Addition of water and triethylamine to the extraction vessel appeared to result in improved SFE recovery of cocaine from drug user and standard reference (fortified) hair. Cocaine was apparently extracted from the hair upon addition of 100 μL of the modifier mixture (15 μL TEA+85 μL H₂O) directly to the hair matrix prior to extraction with CO₂ (i.e., at 110° C., 400 atm), i.e., using a 10 min static and 30 min dynamic extraction period. GC-MS analysis suggested SFE recoveries of cocaine from fortified and drug user hair in the range of 80-90% relative to acid incubation of hair followed by solid-phase extraction. In certain of these disclosed studies (Morrison, J. F.; Sniegoski, L. T.; Yoo, W. J. NIJ Report 601-98, March 1999), SFE with pure CO₂ appeared to compare favorably with methanol rinsing, ethanol rinsing, and combined ethanol/phosphate buffer rinsing in regard to its use as a possible method for decontamination of vapor-deposited cocaine on the hair surface. It was hypothesized in these studies that triethylamine (as the triethylammonium cation) might function to competitively displace cocaine from negatively-charged hair binding sites. Subsequent studies and analyses suggest to the instant inventors that the available data cannot convincingly rule out any of the following possible theoretical metabolic bases for association of drug analytes with hair: namely, (i) electrostatic (positive and negative ionic) interactions of drug analytes with amino acid side chain in hair keratins and associated polypeptides; (ii) hydrophobic interactions with amino acid aliphatic residues, fatty acids and hair sheath lipids; (iii) hydrogen-bonding interactions with H-donor and -acceptor amino acid side chain residues; (iv) van der Waals interactions with one or more components in the hair matrix; and/or (v) physical trapping within the alpha-helical structure of keratin fibrils, i.e., without chemical bonding interactions.

While the nature of the chemical interactions apparently involved in incorporation of drug analytes into hair is at present unknown, studies conducted by the inventors (as set forth in the EXAMPLES section below) have attempted to explore further the possible nature of those interactions by examining the role of SFE modifiers on extraction efficiency. The findings provide basis for the instant methods for SFE-decontamination and extraction of drug analytes from hair samples.

4. Cirimele et al. (Cirimele, V.; Kintz, P.; Majdalani, R.; Mangin, P. 1995. J. Chromatogr. B., 673, 173-181) reported recovery of opiates (codeine, morphine, and 6-monoacetylmorphine), cocaine, and cannabinoids (Δ-9-tetrahydrocannabinol, cannabidiol, and cannabinol) from drug user hair into CO₂ (100° C. and 315 bar) modified with 1 mL of a modifier solution (added to the 7-mL extraction chamber) of methanol-triethylamine-water (2:2:1 v/v) with 10 min static- and 15 min dynamic-extraction times. Extracted analytes were reportedly collected from the effluent by solid-phase trapping on a TENAX trap with subsequent elution using 1.8 niL chloroform. However, it was unclear whether the recovery from trapping and elution were quantitative. According to the authors, GC/MS analysis determined detection limits of 0.3, 0.2, and 0.1 ng drug/mg hair for codeine, morphine, and 6-monoacetylmorphine, respectively, with relative recoveries of 61%, 53%, and 96%, respectively, relative to conventional enzymatic degradation. However, Brewer and colleagues (Brewer, W. E.; Galipo, R. C.; Sellers, K. W.; Morgan, S. L. 2001. Analysis of cocaine, benzoylecgonine, codeine, and morphine in hair by supercritical fluid extraction with carbon dioxide modified with methanol. Anal. Chem. 73: 2371-2376) reported, using GC-MS, cocaine recoveries from SFE that were approximately twice those obtained using conventional acid hydrolysis, raising significant issues about the actual levels of drug analytes associated with the hair matrix that might be extracted if appropriate conditions could be found. Thus, the inventors concluded that conventional SFE extraction methods, trapping and recovery methods as applied to crude hair extracts might not give quantitative recovery of drug analytes.

5. Evaluating the report of Brewer et al. and, specifically, the finding of higher than expected levels of drug analytes, note was taken of the author's off-line methods for derivatizing morphine and benzoylecgonine in the SFE extracts using hexafluoropropanol and pentafluoropropionic anhydride. The inventors concluded that variability in reported drug recoveries among laboratories might reflect differences in derivatization efficiencies of drugs and their metabolites in crude hair extracts rather than differences in extraction efficiencies, and unless more quantitative derivatization methods could be developed GC/MS could not be relied upon as a definite methodology against which to assess optimization of extraction methods and calibration of new detection methods.

6. Confirming the inventors' suspicions that there were significant difficulties associated with detection of drugs and their metabolites in crude hair extracts, Plaut et al. (Plaut, O.; Girod, C.; Staub, C. 1998. Forensic Sci. Int., 92, 219-227) reported the isolation of methaqualone from hair using SFE with CO₂/methanol/water (85:12:3 v/v) at 350 atm and 60° C. and with an extraction time of 40 min. Extracts were analyzed by both GC-MS and capillary electrophoresis (CE). The authors concluded that the SF extracts were too ‘dirty’ for CE analysis (no pre-cleanup SFE steps of the hair were performed), but the selectivity afforded by GC-MS overcame this problem. However, no attempt was made to determine how the drug analytes detected in these experiments might relate to the absolute levels present in drug-user samples. Possibilities also exist for metal ion coordination complexes between the hair matrix and drug and chemical analytes, i.e., particularly where the hair may be dyed and/or treated with shampoos containing di- and trivalent cations.

7. Allen and Oliver (Allen, D. L.; Oliver, J. S. 2000. The use of supercritical fluid extraction for the determination of amphetamines in hair. Forensic Sci.Int. 107: 191-197) reported recoveries of methylenedioxyamphetamine (MDA), methylenedioxymethamphetamine (MDMA), and methylenedioxyethylamphetamine (MDEA) from both spiked and authentic drug-user hair in CO₂/chloroform/isopropanol (90:9:1 v/v) at 3800 psi and 70° C. with a 30 min dynamic extraction period. GC-MS analysis suggested relative recoveries of 84%, 71%, and 77% for MDA, MDMA, and MDEA, respectively for spiked samples, with limits of detection of 0.02, 0.1, and 0.1 ng drug/mg hair, respectively. The method was reported to be linear over a concentration range of 0 to 20 ng drug/mg hair. However, absolute levels in drug-user hair were impossible to determine and recovery from spiked samples is known not to accurately model actual recovery from real samples.

8. In situ derivatization, complexation, or ion-pairing of the analyte to form a less polar and therefore CO₂-extractable species, is one approach for enabling the extraction of polar or even ionic analytes. All of the derivatization, complexation, or ion-pairing reactions can theoretically take place directly in an SFE vessel, i.e., if conditions can be appropriately controlled. The latter methods are referred to herein as “on-line” or “simultaneous” derivatization, complexation, ion pair and SFE. If multiple derivatization steps and losses are to be avoided, judicious choice must be made of reagents in order to facilitate extraction of otherwise insoluble species, and to quantitatively prepare species of derivatives that are suitable for chromatographic analysis without necessity for additional off-line derivatization. Drug analytes from hair appear to be polar, at least in GC/MS analyses.

9. Morrison and Rada (Morrison, J. F.; Rada, A. L. 2000. Abstracts of the 220th National Meeting of the American Chemical Society, Paper No. 412229, Washington, D.C., August) reported a possible combined simultaneous SFE and derivatization of amphetamine, methamphetamine, and MDMA from drug-fortified and spiked hair, i.e., using CO₂ modified with the derivatization reagent mixture of heptafluorobutyric anhydride (HFBA) and ethyl acetate, i.e., 100 mL of each being added to a 60 mg hair sample in a 9-mL extraction vessel. The method was conducted in a stepwise manner within the extraction vessel by first pre-cleaning the hair sample with pure CO₂ and then adding the derivatization reagents. The heptafluorobutyramide derivatives of amphetamine and methamphetamine were recovered from drug-fortified hair at 100° C. and 400 atm, with a 15 min static and 30 min dynamic extaction period. The in situ formation of the drug derivatives enabled the immediate GC-MS analysis of the drug analogs, appeared to obviate the need for additional post-extraction manipulation and derivatization steps.

10. Morrison et al. (Morrison, J. F.; Chesler, S. N.; Reins, J. L. J. Microcol. September 1996, 8, 37-45) reported SFE with radioimmunoassay detection (SFE-RIA) of cocaine residues in drug user hair. Hair was extracted using CO₂ (at 110° C. and 400 atm) modified with 100 mL of a TEA/water modifier mixture (15:85 v/v) added directly to the extraction vessel, i.e., with 10 min static- and 30 min dynamic-extraction periods. Extracts were analyzed for the presence of cocaine using a commercially-available solid-phase RIA kit. Issues unique to the immunochemical analysis of SFE-generated extracts were studied. SFE-RIA analysis of a series of drug-free hair samples established an RIA cut-off value for distinguishing between a negative and presumptive positive cocaine sample at an SF extract concentration of 1.2 ng/mL, or a hair concentration of 0.07 ng/mg. The robustness of the SFE-RIA method was demonstrated by the analysis of a variety of hair samples from both drug users and non-users, and the quantitative SFE-RIA findings correlated well with values obtained by an acid incubation/GC-MS method

In summary, while the apparent speed, relative simplicity and efficiency of SFE suggested possible uses in recovery of a variety of drugs from hair samples, the art also suggested that existing methodology might not offer quantitative recovery, and that GC/MS derivatization and/or detection of drug analytes might also not be optimal. Routine detection of analytes in crude hair extracts by methods other than GC/MS also appeared to be problematic. For instance, while conventional immunoassay formats (e.g., fluorescence-, chemilluminescence- and enzyme-ampliciation-based assays) are capable of detecting analytes at picogram levels, the antibody and/or enzyme proteins, fluorophores, and the like required for the fumctions of these assays are, like the proteins in the hair sample, sensitive to denaturation, acid or base hydrolysis and general destruction or inactivation under the conditions used in SFE extraction. In addition, even neutralized and enzyme-inactivated extract often prove problematic since performance of these immunoassays can be degraded dramatically in the presence of directly interfering substances or materials affecting the background. Many common biosensor assay formats, e.g., those based on changes in electrical potential and/or oscillation, are similarly sensitive to interfering and background substances present in crude extracts. Thus, while immunoassay kits are commercially available for drug screening in urine samples, their usefulness in hair testing may depend in large part on the SFE conditions and methods used for extraction. In addition, any advantage that might be gained from the use of a rapid, simple, sensitive, and inexpensive immunoassay, may be more than offset by lengthy and labor-intensive wet chemical isolation procedures, (i.e., often several hours to 1 day).

While the majority of reported hair drug testing applications of SFE to date have involved GC/MS analysis of the resultant extracts, the unique attributes of the SFE technique make it particularly amenable to coupling with immunochemical analysis, particularly enzyme immunoassays. The coupling of simple immunoassay formats with a rapid and easy-to-use SFE sample extraction procedure offers the potential for an analytical system which efficiently screens out negative samples while accurately identifying presumptively positive samples for confirmatory analysis.

Abbreviations used herein include the following, namely, amphetamine (AM), methamphetamine (MA), cocaine (COC), benzoylecgonine (BZE), cocaethylene (COCA), methylenedioxyamphetamine (MDA), methylenedioxymethamphetamine (MDMA), methylenedioxyethylamphetamine (MDEA), nicotine (NIC), heroin (HER), morphine (MOR), codeine (COD), Δ-9-tetrahydrocannabinol (Δ-9-THC), Δ-9-tetrahydrocannabinol-9-carboxylic acid (Δ-9-THC-acid), dihydroxytetrahydrocannabinol (DH-THC), phencyclidine (PCP), pesticide (as defined below; P), herbicide (as defined below, H); TEA, triethylamine, DEA, diethylamine, MeOH, methanol, DCM, dichloromethane, DCE, dichloroethane, BA, n-butylamine.

As used herein, the terms are intended to have meanings as follows: namely,

“Simultaneous extraction methodology” is intended to mean a method of supercritical fluid extraction comprising one or more steps, but with a single extraction condition in which: (i) two or more different chemical analytes of interest are isolatable from a hair sample in about the same duration of time, in about the same solvent fluid, and in the same chamber, (ii) wherein the subject chemical analytes so isolatable may possess different chemical properties of a cationic and an anionic charge in an aqueous solution, (iii) wherein the hair, the subject fluid and an optional additive are all placed into the same subject extraction chamber; and, (iv) wherein allowance is given during the subject extraction time for physical and chemical changes in the solvent system as may occur e.g. from the interaction of the solvent with the hair sample or with the optional additive included in the chamber.

“Tandem extraction methodology” is intended to mean a method of supercritical fluid extraction comprising two or more steps, with two or more extraction conditions in which: (i) two or more different chemical analytes of interest are isolatable from a hair sample in different durations of time and/or in different solvent fluids, and in one or more chambers, (ii) wherein the subject chemical analytes so isolatable may possess different chemical properties of a cationic and an anionic charge in an aqueous solution, (iii) wherein provision is made for changing the extraction fluid within the subject chamber, or for flowing the partially extracted fluid from a first chamber to a second chamber; and, (iv) wherein allowance is given during the subject extraction time for physical and chemical changes in the solvent system as may occur e.g. from the interaction of the solvent with the hair sample or with the optional additive included in the chamber(s).

“Endogenous drug analyte” is used herein interchangeably with “metabolically incorporated” to mean that the subject drug analyte compound is incorporated within a synthesized cellular product as a result of drug use, blood flow to the cell and chemical association of the drug analyte with the product synthesized by the drug-exposed cell. Representative cellular products so associated with drug analyte include hair keratin matrices, the exfoliative skin keratinous matrices, finger and toe nails, mucus secretions, sputum, biological fluids and the like as disclosed further below in regard to “biological samples”. The subject chemical association of a drug analyte with the cellular product may involve binding of the subject drug analyte to a protein, lipid or fatty acid component in a biological sample (defined below).

“Matrix-associated” is used interchangeably with “metabolically incorporated” and “physiologically incorporated” to mean an “endogenous drug analyte” that is associated with a keratin matrix as a result of a cellular process in a test subject who is a recipient of a drug analyte substance. Representative keratin matrices include hair, finger and toe nails, exfoliative skin and the like. Representative routes by which the test subject may be administered the subject drug analyte include oral, trans-mucosal, trans-dermal, intra-ocular, intra-nasal, intravenous, subcutaneous, intradermal, intramuscular, intrathecal and the like.

“Exogenous drug analyte” is used interchangeably with “environmental contamination” intended to mean drug analyte that is passively associated with hair and as distinguished from metabolically incorporated into hair.

“Isolation” is intended to mean collection of either, or both of, an endogenous drug analyte and an exogenous drug analyte.

“Extraction” is intended to mean the process of isolating and collecting, e.g. onto a solid phase or into a fluid or a vapor, of one or more endogenous drug analytes from a biological sample.

“Drug Analyte” is used herein to refer to a chemical compound present in a biological sample whose determination is of interest to a user of the instant methods, wherein the instant chemical compound, or one or more of its metabolites, produces at least one biological response in a mammal. Representative examples of chemical compounds so capable include pharmaceutical drug compounds, e.g., as set forth in the US Pharmacopia and Physicians Desk Reference, but also other compounds such as those forth in the Merck Index. Representative drug analytes include therapeutic pharmacological drugs, drugs of abuse, and the like including e.g., morphine, opium, cocaine, codeine, amphetamine, methamphetamine, MDMA, THC, β-agonists, steroids and their metabolites. The subject “drug analytes” are distinguished in definition, herein, from potentially toxic chemical compounds and the like which are referred to as “chemical analytes”.

“Chemical analytes” is intended to mean compounds which are not commonly useful as drug substances, but which may instead constitute health risks for man, domestic animals and the environment. The subject chemical analyte may be associated with hair either metabolically, e.g., as a result of ingestion or inhalation, or passively, e.g., as a result of environmental contamination of the hair. Representative examples of the subject chemical analytes include the following: namely, pesticides; herbicides; pollutants; carcinogens; toxic chemicals; environmental microbial toxins such as present in contaminated grains; industrial organic chemicals and plastic polymers comprising risks to human health; organic compounds in animal feeds such as may indicate potential consumption of animal body parts including brain; and the like.

“Modifier” is intended to mean an organic solvent which when added to a supercritical fluid increases the solvent polarity of that fluid. The subject modifiers find use in promoting extraction of polar drug analytes and chemical analytes from a hair sample. According to the instant invention, modifiers may be employed alone or in combination mixtures containing two or more modifiers and optionally containing one or more additives. Representative examples of modifiers include methanol, ethanol, isopropanol, hexane, acetonitrile, ethyl acetate, acetone, dichloromethane, and the like.

“Additive” is intended to mean an organic chemical, including weak acids or bases or ion pairing reagents, which when added to a modifier or a supercritical fluid changes the solvent polarity of the fluid and/or facilitates displacement and removal of analytes from sample matrix binding sites. The subject additives find use in promoting extraction of polar drug analytes and chemical analytes from a hair sample presumably by competing for charged binding sites in carbohydrates and protein side chains. According to the instant invention, additives may be employed alone or in combination mixtures containing two or more additives and optionally containing one or more modifiers. Representative examples of additives useful for elution of basic drug- and chemical-analytes include weak bases such as triethylamine or diethylamine, or ion-pairing reagents including alkyl sulfonic acids such as 1-propanesulfonic acid, 2-propanesulfonic acid, 1-butanesulfonic acid, 1-pentanesulfonic acid, 1-hexanesulfonic acid, 1-heptanesulfonic acid, 1-octanesulfonic acid, 1-nonanesulfonic acid, 1-decanesulfonic acid and sodium dodecyl sulfate (SDS). Representative examples of additives useful for elution of acidic drug- and chemical-analytes include weak acids such as citric acid and ion pairing reagents including alkyl ammonium hydrogen halogen compounds such as tetramethylammonium hydrogen sulfate, tetraethylammonium hydrogen sulfate, tetrapropylammonium hydrogen sulfate, tetrabutylammonium bromide, tetrabutylammonium chloride, tetrabutylammonium dihydrogen phosphate, tetrabutylammonium hydroxide, tetrabutylammonium iodide, tetrapentylammonium bromide, tetrahexylammonium bromide, decamethonium bromide, hexadecyltrimethylanmmonium bromide, hexadecyltrimethylammonium hydrogen sulfate, hexadecyltrimethylammonium hydroxide and the like.

“Chelating agent” is intended to mean a chemical capable of forming a bond with a metal ion. Representative examples of chelating agents include sodium ethylenediamine tetraacetic acid (Na+EDTA), magnesium ethyleneglycol-bis-(β-aminoethyl ether)N,N,N′,N′-tetraacetic acid and the like.

“Derivatizing agent” is intended to mean an agent capable of modifying one or more chemical residues within a drug analyte or a chemical analyte. Representative chemical residues so modified include reactive H-atoms, hydroxyl, carboxyl, amine, amide and the like. Representative derivatizing agents are set forth in the EXAMPLES which follow.

“Catalyst” when used in regard to derivatization and derivatizing agents, is intended to mean a chemical compound which accelerates the subject derivatization reaction without being consumed or altered therein.

“Trap” is intended to mean an equipment component capable of collecting a drug analyte or a chemical analyte extracted from a hair sample in a supercritical fluid. In alternative embodiments, the subject step of collecting said drug or chemical analyte may comprise forming a solution or a dispersion of one or more of the subject analytes in a liquid, or may comprise deposition of one or more of the subject analytes onto or into a solid phase. Representative examples of traps are disclosed in the Examples section, below.

“Test sample” is intended to mean a sample of a biological sample collected from a test subject. While presently preferred embodiments are directed toward biological samples which are keratin matrices, in alternative embodiments, it is anticipated that the methods of the invention will find use with a variety of biological samples, as set forth further below.

“Test subject” is used interchangeably with “subject” to mean humans and their domestic animals.

“Hair collection device” is intended to mean an instrument operated mechanically or electronically to: (i) remove a hair test sample from a test subject; (ii) retain the hair test sample within a chamber; (iii) accomplish each of said removal and retention processes without an operator physically touching the hair test sample. Preferably, the subject hair collection device comprises a device such as that disclosed by certain of the inventors in co-pending U.S. patent application Ser. No. 09/715,879, herein incorporated by reference in its entirety. Most preferably, the subject device is useful as both a hair collection device and as an extraction vessel, as set forth further below.

“Extraction vessel” is intended to mean a fluid reservoir capable of retaining both a test sample and a supercritical fluid in a fluid state under temperature and at pressure. Representative extraction vessels are known in the art, e.g. commercially available hardware available from manufacturers of SFE equipments such as Applied Separations, Inc. and ISCO, and commercially available column and guard cartridge hardware available from chromatography suppliers. Preferably, the subject device comprises at least both of an inlet port and an exit port, both of which ports may be optionally fitted with one or more static or dynamic pressure regulators, flow regulators and/or flow restrictors (e.g., pressure relief and flow controlling valves). Preferably, the subject fluid reservoir is capable of retaining a relatively volume of a supercritical fluid, in this case about 1 mL to about 7 mL, most preferably about 1 mL to about 5 mL. Preferably, the instant extraction vessel also comprises a hair collection device as set forth supra. Most preferably, the instant collection device/extraction vessel is a single use disposable cartridge.

“Machine packet” is used interchangeably with “equipment packet” to mean a container restraining one or more solid or liquid reagents, which container is capable when exposed to a supercritical fluid of establishing fluid communication with the subject supercritical fluid. Representative examples of machine packets include the following: namely, (i) in-line cartridges, columns and the like, wherein the subject fluid communication is e.g. through the two opposing ends of the cartridge or column; (ii) cartridges insertable within an SFE extraction chamber, wherein the subject fluid communication is e.g. through the walls and/or ends of the cartridge; (iii) tea-bag-like porous packages, wherein the subject fluid communication is through the porous walls of the packages; (iv) semi-permeable tea-bags, wherein exposure to the supercritical fluid changes the porosity of the walls of the package; (v) capsules, wherein the contact with the subject fluid communication solubilizes and/or dissolves the wall of the capsule to release the subject reagents; (vi) tablets or pellets, wherein contact with the subject fluid dissolves the tablet or pellet; and the like.

“Immunoassay device” is intended to mean a machine for conducting an automated assay to (i) capture a drug analyte from a supercritical fluid, e.g., onto a solid phase or into a fluid; and (ii) detect the drug analyte so-bound, e.g., as disclosed further in regard to assay formats, below.

“On-Line” when used in regards to methods and equipment for supercritical fluid extraction of drug analytes from a test sample and subsequent detection, e.g., by GC/MS or immunoassay, is intended to mean that the subject SF extraction method, or method step, is conducted in an extraction vessel which is physically linked with, and capable of fluid and vapor exchange with, the subject detection device. For example, the subject extraction vessel internal reservoir/lumen is in sealed fluid and/or vapor phase communication with a detection device, e.g., through tubing equipped with optional valving, flow restrictors, sampling and injection ports, venting ports and the like. A variety of possible different on-line protocols are envisaged according to the invention, including e.g. direct connection of an extraction vessel with a GC/MS detector system or with a dedicated immunoassay device.

“Specific binding partner”, abbreviated SBP, is intended to mean a polypeptide capable of binding to a drug analyte. Representative examples of specific binding partners so capable include antibodies and receptors as they may be isolated from serum and cells of mammals, as well as, from recombinant sources such as prokaryotic and eukaryotic selected and recombinant cell lines. The subject specific binding partners include monoclonal and polyclonal antibodies, as well as, dopaminergic and opiate receptors and the like capable of binding drug analytes. The subject specific binding partners also include fragments of the subject polypeptides, e.g., antibody F(ab)′₂, Fab and complement determining region (CDR) fragments; and, receptor fragments.

“Diagnostic reagent” is intended to mean a reagent suitable for use in a test assay for identifying a drug analyte in a biological sample, e.g. a patient, a human test subject sample or in a sample of food, water or air. The instant diagnostic reagent is suitable for binding a derivatized drug analyte under conditions maximizing the subject binding interaction while minimizing potential cross-reactivity with any plant, mammalian, or avian tissue compounds that may be present in a biological sample. The instant diagnostic reagent additionally, does not bind to a derivative chemical compound. The instant diagnostic reagent commonly contains: (i) a specific binding partner linked to a “signal generating compound”, i.e., a “conjugate” (as defined supra); (ii) one or more buffers, additives, excipients and the like for stabilizing and preserving the subject SBP-conjugate during storage; and/or, one or more substances for promoting the binding activity of the subject SBP-conjugate to a drug analyte in a test assay.

“Biological sample” is used interchangeably with “test sample”, to mean a sample obtained from a living (or dead) organism, e.g., a mammal, fish, bird, reptile, marsupial and the like. Biological samples include tissue fluids, tissue sections, biological materials carried in the air or in water (e.g., pharmaceutical drug compounds and chemicals as they may be bound to living and dead cellular materials) and collectable therefrom e.g., by filtration, centrifugation, and the like. Representative test subjects include humans and their domestic animals. Representative samples, so collected, include food substances, e.g., plant, animal or avian tissues. Representative biological fluids include, e.g. urine, blood, plasma, serum, cerebrospinal fluid, semen, lung lavage fluid, feces, sputum, mucus, water carrying biological materials and the like. Representative biological samples also include foodstuffs, e.g., samples of meats, processed foods, fishes, cereal grains and the like. Biological samples may also include in-situ tissues and bodily fluids (i.e., samples not originally collected for drug testing), for example, the instant methods may be useful in detecting the presence or severity of drug abuse in an emergency room situation where biological samples are collected originally to assay for infection, or underlying disease (e.g., a biopsy specimen). Thus, in different embodiments, the invention finds a variety of uses in different forensics, toxicological and drug testing situations with a variety of different types of biological samples.

“Signal generating compound”, abbreviated “SGC”, is intended to mean a molecule useful in an immunoassay that can be linked to a SBP (e.g. using a chemical linking method as disclosed further below and is capable of reacting to form a chemical or physical entity (i.e., a reaction product) detectable in an assay according to the instant disclosure. Representative examples of reaction products include precipitates, fluorescent signals, compounds having a color, and the like. Representative SGC include e.g., bioluminescent compounds (e.g., luciferase), fluorophores (e.g., below), bioluminescent and chemiluminescent radioisotopes (e.g., ¹²⁵I, ¹⁴C, ³H and the like), enzymes (e.g., below), binding proteins (e.g., biotin, avidin, streptavidin and the like), magnetic particles, chemically reactive compounds (e.g., colored stains), labeled-oligonucleotides; molecular probes (e.g., CY3, Research Organics, Inc.), and the like. Representative fluorophores include fluorescein isothiocyanate, succinyl fluorescein, rhodamine B, lissamine, 9,10-diphenlyanthracene, perylene, rubrene, pyrene and fluorescent derivatives thereof such as isocyanate, isothiocyanate, acid chloride or sulfonyl chloride, umbelliferone, rare earth chelates of lanthanides such as Europium (Eu) and the like. Representative SGC useful in an SBP-conjugate include the enzymes in: IUB Class 1, especially 1.1.1 and 1.6 (e.g., alcohol dehydrogenase, glycerol dehydrogenase, lactate dehydrogenase, malate dehydrogenase, glucose-6-phosphate dehydrogenase, glyceraldehyde-3-phosphate dehydrogenase and the like); IUB Class 1.11.1 (e.g., catalase, peroxidase, amino acid oxidase, galactose oxidase, glucose oxidase, ascorbate oxidase, diaphorase, urease and the like); TUB Class 2, especially 2.7 and 2.7.1 (e.g., hexokinase and the like); TUB Class 3, especially 3.2.1 and 3.1.3 (e.g., alpha amylase, cellulase, β-galacturonidase, amyloglucosidase, β-glucuronidase, alkaline phosphatase, acid phosphatase and the like); IUB Class 4 (e.g., lyases); IUB Class 5 especially 5.3 and 5.4 (e.g., phosphoglucose isomerase, trios phosphatase isomerase, phosphoglucose mutase and the like.) The subject signal generating enzymes may be coupled to a non-antibody SBP, e.g., a recombinant drug analyte receptor, or to a second binding partner used in combination with a SBP, e.g., a different second antibody SBP, e.g., in a sandwich immunoassay format The subject SGC share the common property of allowing detection and/or quantification of a drug analyte in a test sample. Preferably, the subject signal generating compounds are detectable using a visual method, a spectrophotometric method, an electrical method (e.g., a change in conductance, impedance, resistance and the like), or, a fluorescent detection method.

“Solid phase”, as used herein, is intended to mean a surface to which one or more drug or chemical analytes, or immunoassay reactants may be attached electrostatically, hydrophobically, or covalently. Representative solid phases include e.g.: nylon 6; nylon 66; polystyrene; latex beads; magnetic beads; glass beads; polyethylene; polypropylene; polybutylene; butadiene-styrene copolymers; silastic rubber; polyesters; polyamides; cellulose and derivatives; acrylates; methacrylates; polyvinyl; vinyl chloride; polyvinyl chloride; polyvinyl fluoride; copolymers of polystyrene; silica gel; silica wafers glass; agarose; dextrans; liposomes; insoluble protein metals; and, nitrocellulose. Representative solid phases include those formed as beads, tubes, strips, disks, filter papers, plates, solid phase extraction (SPE) sorbent phases available from suppliers of sample preparation and chromatography accessories, and the like. Filters may serve to capture a drug or chemical analyte e.g. from a supercritical fluid, or act by entrapment, or act by covalently-binding SBP onto the filter. A solid phase capture reagent for commercial distribution to a user may consist of a solid phase (supra) coated with a “capture reagent” (below), and packaged (e.g., under a nitrogen atmosphere) to preserve and/or maximize binding of the capture reagent to a drug analyte in a biological sample.

“Capture reagent” is intended to mean an immobilized SBP capable of binding a drug analyte. The subject capture reagent may consist of a solution or SBP modified so as to promote its binding to a solid phase, or as an SBP already immobilized on a solid phase, e.g., immobilized by attaching the SBP to a solid phase (supra) through electrostatic forces, van Der Waals forces, hydrophobic forces, covalent chemical bonds, and the like (as disclosed further below.) Representative examples of SBP-capture reagents are disclosed in the EXAMPLES, section below, and include mobile solid phase SBP-capture reagents such as SBP immobilized on movable latex beads e.g. in a latex bead dipstick assay.

“Detect reagent” is intended to mean a conjugate containing an SGC linked to a SBP. Representative examples of the instant detect reagents include SGC-SBP; covalently-linked complexes of a first SBP and a second SBP, e.g., an SBP-#1 cross-linked with an SBP-#2; and the like. The subject detect reagents include mobile solid-phase detect reagents such as movable latex beads in latex bead dipstick assays.

“Specificity”, when used in the context of an assay according to an embodiment of the invention, is intended to mean that the subject assay, as performed according to the steps of the invention, is capable of properly identifying an “indicated” percentage of samples from within a panel of biological samples (e.g., a panel of 100 samples). The subject panel of samples all contain one or more drug analytes (e.g., positive control samples purposefully contaminated with a drug analyte). Preferably the subject “indicated” specificity is greater than 85%, (e.g., the assay is capable of indicating that more than 85 of the 100 samples contain one or more drug analyte), and most preferably, the subject assay has an indicated specificity that is greater than 90%.

“Sensitivity”, when used in the context of an assay according to an embodiment of the invention, is intended to mean that the subject assay, as performed according to the steps of the invention, is capable of identifying at an “indicated” percentage those samples which contain a drug analyte from within a panel of samples containing both positive controls (supra) and negative controls (i.e., lacking drug analyte.) Preferably the subject “indicated” sensitivity is greater than 85% and most preferably greater than 90%.

“Background”, when used in the context of an assay according to an embodiment of the invention, is intended to mean the level of signal detected in a negative control sample, wherein nonspecific reactant may contribute to signal generation at a level higher than that observed in a buffered solution. Representative background in biological samples may contribute to uncertainty in interpreting a test result which may sometime expressed as a measurement of a degree of confidence in a test result.

“Interfering substance”, when used in the context of the instant methods, is intended to mean a substances which may prevent with the proper performance of the instant assay when it is present in the assay. Representative examples of substances which may so interfere, (i.e., also known in the art as confounding substances, and the like), include materials present in biological samples such as animal lipids and complex carbohydrates, plant lignins, cellulosic materials and the like, as well as, specific enzyme inhibitors or substrates that react directly with signal generating compounds.

“Substantially purified” is used herein to refer to a preparation that contains a SBP that is enriched greater than about 10-fold to about 25-fold, preferably greater than about 26-fold to about 50-fold and most preferably greater than about 100-fold from the levels present in a source material. The subject preparation also preferably contains less than about 10% impurities, and most preferably less than about 5% impurities detectable e.g. by either SDS-PAGE or reverse-phase HPLC.

“Drug test assay” is intended to mean an experimental procedure performed on a test sample using a medical diagnostic assay device for the purpose of detecting the possible presence or amount of an analyte in the test sample. The subject test procedure may e.g., be performed on-site, i.e., at the employer facility, or alternatively, in a diagnostic laboratory. Representative examples of the subject drug test assay include a variety of enzyme-linked immunoassay devices, radioimmunoassay methods and methods employing gas chromatography (GC) and mass spectrometry (MS), e.g., tandem GC/MS, as are known in the art or as may be later developed and implemented as accepted drug testing methodologies. As an optional feature of the present invention, consumer data regarding the performance of a diagnostic laboratory may be collected including eg., responsiveness, confidentiality, consumer confidence, performance results reported on control validated “spiked” drug-positive samples and the like. As an additional optional aspect, data relating to the technical performance of a laboratory test, manufactured diagnostic test device, or other assay methodology, may be collected, stored and used to make calculations such as those known to those skilled in the diagnostic arts, e.g., accuracy, precision, specificity, sensitivity, “false positive” and “false negative” rates, and the like. It is anticipated that the latter performance data and calculations, as part of the instant invention, may be made available to users so that a DER, and/or MRO, may more readily make informed decisions with regard to the suitability and performance characteristics of particular assays and diagnostic test laboratories..

“Screening assay” is used to mean an assay procedure that is performed on a biological specimen to qualitatively determine whether drug analyte is present. Representative screening assays include a variety of immunoassays as set forth supra. Commonly, screening assays will not involve a molecular determination of the chemical species of drug analyte present in a biological sample.

“Confirming assay” is used to mean an assay procedure that is performed on a biological specimen to determine the nature of the chemical drug analyte entities therein. Representative confirming assays include GC/MS, tandem GC/MS, ion trap GC/MS methodologies and the like. Commonly, a confirming assays may be performed after a positive result is recorded in a screening assay.

“Validating assay” is used to mean an assay procedure performed on a biological sample for the purpose of detecting the possible adulteration of a that biological sample. The subject test procedure may, e.g., be performed on-site or in a diagnostic laboratory. Representative validation assays include those testing for the concentration of a normal urine constituent, eg., creatinine or specific gravity, to determine whether a test sample has been diluted as well as assays specifically designed to detect adulterants.

“Sample collection”, when used in regard to a test sample, is intended to mean the process of obtaining a test biological sample from a drug test subject.

“Trap” when used in regard to collection of a supercritical fluid containing an extract, is intended to mean a container in supercritical fluid communication with an SFE extraction chamber. Preferably, the subject trap is connected through a restrictor with the subject extraction chamber

“Restrictor” when used in regard to supercritical fluid extraction, is intended to mean a device for restricting the flow of supercritical fluid and maintaining supercritical conditions upstream of its position e.g., from the outlet of an extraction chamber. In use, the subject restrictor determines the rate of flow of fluid from the subject extraction chamber into a trap. Representative examples of restrictors include variable orifice needle valves, capillary tubing of various bores, lengths and materials construction, e.g., stainless steel, thermoplastics, fused silica and the like, as well as in-line columns filled with packing. Preferably, the subject restrictor is of a material and configuration that allows heating. Heating is preferably to a temperature effective to prevent freezing and plugging of the restrictor during transit of the supercritical fluid, preferably, the subject restrictor is heated to a temperature that is about 5° C. higher than the extraction temperature.

“In-line” when used in regard to a component of a n SFE apparatus, or a step in an SFE method, is intended to mean that the subject component or step is conducted in a manner such that it is in continuous fluid communication with the supercritical fluid within the subject SFE apparatus.

“In situ” when used in regard to a physical location at which a step in a supercritical extraction method is conducted, is intended to mean that the subject step is performed within the extraction chamber containing a sample (eg., hair) and a supercritical fluid. In situ does not refer to a temporal sequence and the subject step may be performed before, during or after the extraction of a drug- or a chemical-analyte from a hair sample.

“β-agonist” is intended to mean phenylethanolamines possessing a common β-hydroxyamino side chain group. Representative examples of the subject β-agonists include clenbuterol, salbutamol, terbutaline, fenoterol and the like.

“Pesticide” is intended to mean a chemical analyte toxic for an insect. Representative examples of the subject pesticides include the following: namely, organochlorine compounds (OCCs) including chlorophenols,, polychlorinated biphenyl compounds (PCB), sulfonylureas, and the like such as, DDT, atrazine, dieldrin, carbofuran, 2,4-(dichlorophenoxy)acetic acid (2,4-D), 2,4-(dichlorophenoxy)butanoic acid (2,4-DB), 2,4-dichlorophenol (2,4-DCP), 4-nonylphenol (4-NP), chlorophos, methamidophos, dichlrovos, methamidophos, mevinphos, acephate, tetrahydrophthalimide, pentachlorobenzyne, o-phenylphennol, omethoate, propoxur, diphenylamine, chloropropham, trifluralin, phorate, hexachlorobenzyne, dicloran, dimethoate, carbofuran, atrazine, quintozene, lindane, terbufos, diazinon, chlorothalonil, disulfoton, phosphamidon, vinclozolin, parathion-methyl, carbaryl, malathion, chloropyrifos, aldrin, dacthal, parathion, dicofol, captan, methidathion, disulfoton sulfone, endosulfan, fenamiphos, myclobutanil, endosulfan, ethion, propargite, iprodione, phosmet, methoxychlor, phosalone, azinphos-methyl, permethrin, cyfluthrin, cypermethrin, fenvalerate, antrhacene, chrysene and, where applicable, their glucosides and metabolites such as 1,1 -dichloro-2,2-di(4-chlorophenyl)ethylene (DDE), 1,1,1-trichloro-2,2-bis(4-chlorophenyl)ethane, heptachlor, chlorpyrifos, chlordane, endrin, dichloran, tecnazene, diazinon and the like.

“Herbicide” is intended to mean a chemical analyte toxic for a plant. Representative herbicides include e.g. alachlor, bromacil, hexazinone, metolachlor, metribuzin and the like.

“Steroids” is intended to mean a compound having a tetracyclic (cyclopentanophenanthrene) nucleus. Representative examples of the subject steroids include aldosterone, androsterone, cholecalciferol, testosterone, nortestosterone, methyl-testosterone. cortisol, cortisone, ergocalciferol, estradiol, estrone, lanosterol, progesterone and their metabolites, glucuronides and the like.

Embodiments of the invention provide for testing of biological samples either (i) in an on-site screening assay device, i.e., to identify the small number of possibly positive samples needing additional testing, and/or (ii) in a laboratory-based assay system. Embodiments of the invention produce testing methods useful in screening assays, confirming assays and validating assays.

Embodiments of the invention provide environmentally friendly and cost-effective extraction methods based on use of supercritical fluids (SFs) and hair samples to achieve rapid and reliable isolation of drug analytes from hair samples. In other embodiments, methods are provided for SF extraction and integrated drug isolation, derivatization, and detection steps. The instant methods provide an analytical platform which efficiently screens out negative samples while accurately identifying presumptively positive samples for confirmatory analysis. The instant extraction methods optionally produce drug-derivative analytes suitable for detection by a variety of fluid phase immunoassay formats, solid phase assays and GC/MS techniques.

Embodiments of the invention provide rapid, safe, cost effective, high throughput, environmentally friendly extraction of drug analytes from samples of drug-user hair.

In other embodiments, the invention provides methods for efficient extraction of drug analytes from human drug-user hair at high absolute recoveries. The instant methods take optimal advantage of the favorable mass transport properties of supercritical fluids, their gas-like high diffusivity and low viscosity to achieve rapid and efficient extraction. The instant methods also take advantage of Boyle's gas law variables, i.e., Pressure, Volume and Temperature, to change the density, and thus solvation power, of the SF extraction solvent in a manner effective to achieve selective extraction of drugs of abuse from drug-user hair.

In yet other embodiments, the invention provides additive modifiers for SFE which selectively alter the composition of the supercritical fluid in a manner effective to achieve reproducible extraction of drug analytes from drug user hair sanples.

In still other embodiments, the invention provides sequential SFE methods for first decontaminating a hair sample to remove exogenous environmental drug analytes from the surface of the hair under a first set of SFE conditions, then followed by a second set of SFE conditions that achieve optimal extraction of drug analytes from drug user hair.

Embodiments of the invention also provide SFE extracts of hair suitable for use in routine immunoassay formats including laboratory-based assays and on-site kit assay formats for use in commercial and doctor's office environments.

In other embodiments, the invention provides tuneable methods wherein extraction conditions are varied to differences in hair samples obtained from different individuals.

In other embodiments, the invention provides SFE methods that allow rapid post-extraction concentration of drug analytes from a crude hair extract.

In other embodiments, the invention provides SFE methods that minimize the cost—and environmental hazards associated with present day removal and disposal of liquid solvent wastes generated in hair testing for drugs of abuse.

In other embodiments, the invention provides an automatable SFE method suitable for use in commercial test equipment.

In other embodiments, the invention provides improved methods for derivatization of drug analytes in crude hair extracts prior to detection by GC/MS methods.

In other embodiments, the invention provides methods for in-situ derivatization of drug analytes in supercritical fluids.

In other embodiments, the invention provides methods for simultaneous SF extraction and derivatization of drug analytes in drug user hair. The instant methods utilize derivatizing agents stable and chemically active in SF extraction solvents under conditions of temperature, pressure and volume suitable for use in SFE.

In other embodiments, the invention provide methods for in-situ simultaneous SF extraction and derivatization in a manner effective to increase the absolute levels of drug analytes extracted from drug user hair by changing the chemical composition of those analytes, i.e., in-situ, in a manner effective to increase their extractability. The instant methods take advantage of changes in polarity and extractability that accompany derivatization of drug analytes, i.e., the instant derivatized drug analogs are less polar and more readily extractable into nonpolar SF solvents such as CO₂.

Modifiers and Additives: Embodiments of the invention also provide hair matrix modifier compositions suitable for use in SF extraction of drug analytes from drug user hair samples. The instant hair matrix modifiers and additives facilitate removal of analytes from binding sites in hair by disrupting chemical bonds involved in binding of drug analytes to hair. Representative examples of chemical bonds so disrupted include covalent, ionic, hydrophobic, hydrogen-bonding and/or Van der Waals forces.

Derivatizing Agents: In other embodiments, the invention provides more efficient and cost-saving methods for simultaneously extracting and derivatizing samples for GC/MS analysis. The instant methods have fewer manipulative steps, eliminate post-extraction processing and offer less opportunity for switching of samples, inadvertent operator error or purposeful interference. The instant methods are amenable to automation. The crude extract derived from the instant methods is suitable for direct injection into detect assay, e.g., GC/MS. Thus, in yet other embodiments, the invention provides methods for a closed system in which a sample is subject to SF extraction and derivatization and then, without operator handling, direct automated injection into a detection assay such as GC/MS, capillary electrophoresis and the like.

Equipment Components: SFE equipment components useful according to the instant methods are commercially available as follows: namely, e.g., from ISCO, e.g., Model SFX-220 or -3560 (ISCO, Lincoln, Nebr.); Hewlett-Packard, e.g., extraction trap module #7680A; Suprex or Lee Scientific Instruments, e.g., Suprex SFC/200A supercritical fluid chromatograph; Keystone Scientific, e.g., SFE vessel; Applied Separations, e.g., the “Spe-ed SFE” system (Applied Separations, Allentown, Pa.). In addition, a variety of components are commercially available for certain assemblies, as follows: namely, e.g. Browlee Labs. micropumps for mobile phase delivery and pressure control (Applied Biosystems, Santa Clara, Calif.), back pressure regulators, manometric pressure monitors and software controllers (e.g., Gilson), electrically actuated high-pressure valve injection loops (Valco Instruments, Houston, Tex.), GC ovens (e.g., Perkin-Elmer, Hitachi and other), extraction chambers (e.g., HPLC column and guard column hardware available from chromatography suppliers), protective filters and cartridges (e.g., SPE cartridges, C18 cartridges and filters, Hydromax cartridges and the like), outlet pressure restrictors, flow restrictors (e.g., Polymicro Technologies, Phoenix, Ariz.), degassers and traps, post-extraction-chamber solvent-flush trap pumps and the like, as known to those of skill in the art for use in supercritical fluid extraction and temperature and pressure controlled collection of analytes. Equipment for confirming extraction by UV spectroscopy, therm-ionic detection, flame ionization detection, GC/MS, diode array detection and the like is commercially available.

Hair Samples. Assays according to the instant methods are performed using hair samples comprising about 1 mg to about 50 mg by weight, preferably about 5 mg to about 20 mg. The hair sample is loaded into an extraction vessel in the supercritical fluid extraction instrument; in a presently preferred embodiment, the extraction vessel comprises a hair collection device such as that disclosed by certain of the inventors in co-pending U.S. patent application Ser. No. 09/715,879 (filed Nov. 17, 2000 and incorporated herein by reference in its entirety). The instant hair collection device preferably serves as both the collection and the extraction vessel, i.e., it is preferably directly inserted into the heated zone (chamber) of an extraction instrument.

Immunoassay Reagent Components: In yet other embodiments, the invention provides immunoassay reagents, assay, test kits and the like for detecting derivatized-drug analytes in SF extracts of hair samples. The instant reagents involve antibody binding partners which are specific for derivatized-drug analytes, which are specific for the subject drug-derivative complex and not capable of binding to a derivative chemical compound.

Immunoassay Methods: Embodiments of the invention also provide reagents usefuil in assay formats for identifying drug analytes in a variety of different types of biological samples. Representative assay formats useful for detecting drug analytes include enzyme-linked solid-phase absorbent assays (e.g., ELISA), radiolabeled binding assays (e.g., RIA), fluorescence binding assays (e.g., FIA), time-resolved fluorescence assays (e.g., TRF), as well as, sandwich- and enzyme-cascade assay formats. Illustrative methods, as may be adaptable from the immunoassay art for use in the subject assays include: homogeneous assay formats; heterogeneous assay formats; competitive assay formats; non-competitive assay formats, enzyme-linked solid phase assay formats, fluorescence assay formats, time resolved fluorescence assay formats, bioluminescent assay formats and the like. The instant assay formats differ from the former assays in the art in their specific detection of derivatives of drug analytes, i.e., not drug analytes per se. Thus, the instant diagnostic assay formats and diagnostic reagents differ from those in the art by virtue of their ability to detect endogenous and exogenous drug analytes as they are found in situ after SF decontamination and derivatization and/or SF extraction and derivatization.

Thus, the instant diagnostic assay formats and diagnostic reagents include those usefuil for detecting endogenous and exogenous drug analytes as they are found in situ after SF extraction and derivatization.

The instant assay methods include those having a step effective to simultaneously accomplish binding and signal generation as an analyte binds to a SBP, i.e., a “simultaneous” or “homogeneous” assay format. The instant methods also include “heterogeneous” drug-binding assay formats, including one or more steps for separating a “bound” from a free analyte and then generating a signal. The instant methods also include those having a step in which analyte is added to compete with the binding of a labeled ligand to an SBP, i.e., a competitive binding (or indirect) assay format, or alternatively, in which binding of an analyte to a SBP is detected by adding a second SBP having signal generating compound, i.e., a non-competitive (or direct) assay format. Illustrative methods for separating “bound” analyte, ligand or SBP from “free” include filtration and column separation, magnetic separation , as well as attaching one or more of the reactants to a solid phase. Illustrative assay methods for detecting a signal generating compound commonly include: using an enzyme as a SGC that converts a substrate to a visually or spectrophotometrically identifiable product), or alternatively, exciting a fluorescent SGC coupled to an SBP so that a detectable signal is emitted, e.g. at a different wavelength, e.g. fluorimetric analysis.

Commonly, coating polystyrene (e.g., 96-well Dynatech-Immulon II, Nunc, or similar plates) with SBP at a concentration of about 1 mg/ml in a carbonate buffer at pH 8 for about 16 hours results in binding of about 20 to about 150 μg/well to the solid phase.

Embodiments of the invention include, SBP solid-phase assay formats having at least the following three steps: namely,

In a first step, one or more drug analytes in a biological sample are extracted into a supercritical fluid containing both a modifier and/or additive and an optional derivatizing compound under conditions suitable for formation of a chemical bond between the extracted drug analyte and the derivatizing compound;

In a second step, a drug analyte-derivative so-formed in step 1, is ‘captured’ by binding, (e.g., involving covalent, ionic and/or hydrophobic interactions), to an inert solid phase, or alternatively, to a SBP that is attached electrostatically (or covalently) to a solid phase;

If necessary unbound materials (e.g., constituting possible background or interfering substances) are removed by washing; and,

In a third step, the bound drug analyte-derivative from step-2 is detected by reacting it with a SBP “detect reagent” having a signal generating compound, whereafter binding of the SBP-SGC detect reagent may be detected by identifying the presence or amount of the SGC, e.g., by measuring fluorescence, adding substrate to detect enzyme activity and the like.

Other steps in the instant diagnostic assay formats may include one or more optional steps, i.e., before step-1 (supra), for pretreating a biological sample (e.g. a lung lavage, saliva, sputum or mucus sample) with a mucosidase, a detergent (e.g., TWEEN-20), a DNAase, a collagenase and/or a protease. The preferred pretreatment step decreases the viscosity in a sample and improves the automated sample handling or solubility of the sample; and/or, the pretreatment denature endogenous confounding or interfering substances and improves assay performance.

“Uniform assay format” is intended to mean that the molecule capable of capturing (e.g., SBP #1) a drug analyte, e.g., on a solid phase, and the molecule capable of detecting the captured drug analyte (i.e., SBP #2) are the same SBP, e.g., SBP#1 and SBP#2 are both monoclonal antibody. It is intended within this definition that different forms of the non-antibody SBP (as defined by SBP#1, SBP#2, and SBP#3, supra) may be used for the capture reagent and the detect reagent, e.g., recombinant dopaminergic receptor D1, D2 or D3 or recombinant opiate receptor polypeptide as SBP#1 and a monoclonal antibody as SBP#2.

“Mixed assay format” is intended to mean that in a first step of the assay the molecule capable of capturing a drug analyte compound is different than the molecule capable of detecting the captured drug analyte in a second step, e.g., an antibody SBP with covalently bound biotin in the first step and a streptavidin capable of binding the biotin in the second step.

“Combined simultaneous assay format” is intended to mean simultaneous detection of more than one drug analyte in a single assay format, e.g., simultaneous detection of two drug analytes by detecting the binding of two different SGC compounds. In one representative example, the two different SGC compounds used for simultaneous detection preferably have different fluorescence excitation- and emission-profiles. Preferably, the subject excitation- and emission-profiles are only partially overlapping and most preferably the subject profiles are non-overlapping.

In certain other embodiments, the invention provides diagnostic reagents containing SBP that are detect reagents specific for derivatized drug analytes. The instant detect reagents contain one or more signal generating compounds conjugated to an SBP. Representative methods for covalently linking SGC to SBP include those using hetero-bifunctional cross-linking reagents that are reactive with carbonyl, aldehyde, carboxyl, amino, disulfide and thiol groups of amino acids, e.g., carbodiimide, N-hydroxy succinimide, N-succinimidyl-3-(2-pyridyldithio)propionate (SPDP), maleimide, succinimidyl-pyridylthioproprionate, m-maleimidobenzoyl N-hydroxysuccinimide ester, succinimidyl pyridylthiopropionate, and the like. Methods for linking SGC fluorophores to SBP include, e.g., encouraging electrostatic interactions of the subject SBP with the fluorophore by placing the SBP in a buffer having a Ph below its isoelectric point (e.g., Ph 2.5-3). Methods suitable for linking phycobiliproteins to SBP are disclosed in Stryer et al. U.S. (patent Ser. No. 5,055,556).

The instant SBP-SGC conjugates (supra) are prepared for commercial distribution as detect reagents, preferably by solubilizing them in one or more buffer solutions and then dispensing them into reagent bottles, packages and the like; or, alternatively, by lyophilizing them and dispensing them as powders into reagent packages. The subject buffer solutions may include additives (e.g., stabilizers), emulsifiers (e.g., detergents), and the like for preserving the activity of the instant SBP-conjugate during storage; or, for promoting the binding activity of the instant SBP for a drug analyte in the instant assays. Examples of agents that may be used to promote the subject binding interactions include additives that decrease total fluid volume in an assay (e.g., polyethylene glycol, sucrose and the like); and, agents that promote interactions by provide an electrostatic surface in solution (e.g., dextran, polystyrene beads, polyacrylate beads, and the like.)

Embodiments of the invention provide assay formats including competitive and non-competitive, direct and indirect, quantitative and non-quantitative assays including ELISA (enzyme-linked immunosorbent assay), RIA (radioimmunoassay), FIA (fluorescence immunoassay), TRF (time resolved fluorescence) and cascade assay formats (supra).

“Cascade assay formats” is intended to mean detecting a drug analyte compound through a process: wherein, a first signal generating compound (i.e., SGC #1) produces a product that can be utilized by a second SGC #2 to produce a product which e.g. can be utilized by a third SGC #3. The subject cascade of products from SGC #1-3 results in amplification which results in a greater overall signal that could be achieved by any single SGC.

Immunoassay Kits: Embodiments of the invention also provide kits useful for identifying drug analytes in a biological sample. A representative kit contains the following: namely, one or more reagent packages at least one of which contains a SBP-conjugate; an assay buffer; an optional assay surface, e.g., a tray, a vessel, or dipstick; a set of instructions; and one or more optional assay calibrators or reference compounds (e.g., a positive and negative control). In one presently preferred embodiment, a kit contains reagent packages containing: (i) one or more optional pre-treatment solutions for reducing viscosity of biological samples containing mucus, and the like, and for reducing background and interfering substances; (ii) one or more reference calibrator solutions (e.g., one or more drug-analyte-derivatives); (ii) one or more optional modifier solutions suitable for use in SFE; (iv) one or more SBP-conjugates (e.g., SBP-FITC, SBP-enzyme and the like); (v) one ore more optional assay buffers and/or wash buffers (e.g., assay buffer containing PEG and/or detergents that promote binding between SBP and a drug analyte compound); (vi) one or more optional enzyme substrate solutions; (vii) one or more optional blocking buffers for reducing nonspecific background (e.g., solutions containing BSA or milk proteins); and (viii) one or more optional solid phase reaction surfaces upon which, or in which, the assay may be conducted (e.g., microtiter plates, dipsticks, strips, and the like.)

### EXAMPLE 1

### On-Line (In Situ) Derivatization/SFE

Laboratory-Based Method for Integrated Drug Extraction and Derivatization:

Summary Overview: In the analysis of drug compounds, including drugs-of-abuse and pharmaceuticals, it may often prove advantageous to perform chemical derivatization reactions prior to chromatographic analysis in order to convert the target analytes to analogs that have different chemical properties, e.g., analytes that are more amenable to the particular analytical detection technique chosen for detection and quantification of the compounds (i.e., typically, gas chromatography (GC) or gas chromatography-mass spectrometry(GC-MS)). Analytical derivatization prior to, or during, SFE accomplishes formation of one or more of the following: namely,

1. a derivative which is more extractable than the parent compound;

2. a derivative which is more stable than the parent compound;

3. a compound which through the introduction of functional groups is rendered suitable for subsequent chromatographic methods such as GC and GC-MS and which functional groups are recognized by selective detectors (GC) and/or direct fragmentation to produce readily identifiable fragment ions (GC-MS);

4. a compound that overcomes adsorption problems leading to poor extraction kinetics and/or tailing during extraction and chromatography;

and

5. a compound with improved extraction separation efficiency, e.g., expressed as percentage recovery.

Analytical derivatization reactions employed most commonly for derivatization of drug analytes and chemical analytes may be grouped into three major reaction categories: namely, acylation (acetylation), alkylation, and silylation. Analytes containing functional active hydrogen groups, i.e., —NH, —OH, —COOH, and —SH, are commonly converted through their reaction with acetylating, alkylating, or silylating agents to analogs, e.g., amides, n-alkylamides, esters, thioesters, ethers, thioethers, n-alkylamines, sulphonamides and trimethylsilyl derivatives. The latter analogs often exhibit improved extraction kinetics relative to the parent compounds. Unlike present day methods where derivatization is performed on a sample extract after the steps involved in isolation of a target drug by e.g. liquid-liquid or solid-phase extraction techniques (LLE and SPE, respectively), the instant methods involve derivatization in situ during extraction in a supercritical fluid. Unlike present methods where an extract is typically evaporated just to dryness prior to the addition of one or more derivatization reagents, the instant methods involve derivatization in supercritical fluid. Unlike present methods where derivatization is accomplished by additional time and labor intensive steps of heating, e.g. to accelerate the derivatization reaction, evaporation and/or back-extraction to remove excess reagents or reaction byproducts, the instant methods accomplish rapid in situ derivatization in a dynamic flow mode where the reaction product may be amenable to in-line analysis e.g. by GC/MS. Analyte derivatization performed according to present day methods are labor intensive, tedious, time-consuming and involving multiple manipulations that are not readily automated each of which introduces the potential for significant analyte losses and errors. The instant methods solve these problems.

According to the instant methods, set forth below, the processes of extraction and derivatization is accomplished simultaneously in situ during SFE, i.e., with resulting improvements in isolation yields, kinetics of extraction, stabilizing of chemical structures and formation of derivatives immediately available for use in GC or GC-MS detection and confirmation assay methodologies. The following benefits are realized according to the instant methods when analyte derivatization is directly coupled with supercritical fluid extraction: namely,

1. Extraction and derivatization are accomplished simultaneously, i.e., during the same time period, in situ, i.e., in the sample extraction chamber. According to alternative embodiments, the instant derivatizing reagents are either introduced directly into the SF extraction solvent or placed into the extraction chamber along with the hair sample, e.g., using equipment packets as set forth supra, and illustrated below;

2. Derivatized drugs are less polar than the underivatized parent polar compounds and are therefore more readily extracted into relatively nonpolar supercritical fluids like CO₂;

3. Derivatization reagents function as “matrix modifying agents”, i.e., agents which chemically modify the hair glycoprotein and complex carbohydrate matrix resulting in disruption of chemical bonds involved in binding of drug and chemical analytes. As a result, extraction of the drug analytes from hair can occur more rapidly, more reproducibly, and with improved yields;

4. Under the instant methods of supercritical fluid derivatization at elevated temperature, derivatization reactions proceed more rapidly, with better efficiency and to more complete endpoints;

5. Extraction of drug and chemical analytes from hair in a derivative form ready for direct detection by GC/MS and/or immunoassay analysis obviates the need for additional post-extraction manipulation and derivatization steps;

6. Isolation of drug derivatives suitable for use in both screening assays and confirmation assays minimizes sample preparation time, losses and potential chain of custody issues where legal proceedings may be involved; and,

7. The instant methods allow direct in-line interfacing of SFE extraction and GC and/or GC/MS detection in closed system, e.g., with direct coupling of SFE extractant fluid to either of the following alternative embodiments, namely, (a) solid phase trapping with subsequent off-line elution and GC or GC/MS analysis; (b) solution trapping with subsequent off-line GC or GC/MS analysis; (c) in-line trapping and direct in-line elution and in-line GC or GC/MS analysis; or (d) direct detection in MS or GC/MS without intervening off-line sample handling or manipulation.

General Features of Derivatization: The instant methods takes advantage of derivatization chemistry, and preferably acylation, to simultaneously improve extraction and derivatization efficiencies, and decrease analysis time, i.e., with cost savings. While the individual derivatization reactions vary somewhat, i.e., depending upon the chemistry of the particular drug analyte, those skilled in the art will recognize that a variety of modification can be made to the chemistry without departing from spirit and scope of the disclosure. Particularly, alternative embodiments share the common features of simultaneous in situ derivatization and SFE. General instructions are disclosed as follows: namely, the chemical reagents and conditions employed in the instant approach are dependent upon the particular derivative desired, which is in-turn based on a variety of factors, including at least: (i) the reactive functional groups present in the target analytes; (ii) the ultimate detector system to be employed for the possible off-line approaches, e.g., GC/MS, NMR, IR etc.; (iii) the desirable, or desired, fragmentation ions for detection e.g., in MS identification; and, (iv) the reactivity and thermal and hydrolytic stability of desired resultant derivatives; (v) the chemical nature of any possible reaction byproducts (e.g., volatility, acidity, reactivity).

### EXAMPLE 2

### Derivatization of Drug Analytes Having Reactive Hydrogen Atoms

Reactive hydrogens groups include e.g., hydroxyl, amine, amide and thiol groups. The subject analyte compounds containing these reactive hydrogen groups may have low extractability because of their tendency to form hydrogen bonds with carbohydrates and protein side chain residues in the hair matrix. Chemical derivatization methods are known for masking hydrogen group reactivity including formation of alkyl, silyl and/or acyl derivatives, Field, J. A. (J. Chromatogr. A 785: 239-249, 1997); Knapp, D. R. (Handbook of Analytical Derivatization Reactions, 1979), both of which disclosures are incorporated herein by reference in their entirety.

Unfortunately, existing derivatization reactions are not universally applicable to hair matrices, or to drug analytes and metabolites, or to simultaneous extraction of two or more drug analytes and their metabolites from hair, or to drug analytes as they are bound in the hair glycoconjugate matrix and modifications may be required according to the instant disclosure with consideration being given to the following issues: namely, (i) the abundance of active hydrogen atoms in the hair matrix; (ii) the intrinsic chemical instabilities of certain drug and other analytes and their metabolites in hair; (iii) the chemistry of derivatizing agents in CO₂ under the particular supercritical conditions useful in hair extraction, e.g., effects of modifiers, additives and water in partial fluid phase, pH, CO₂/carbonic acid phases and the like as needed to extract analytes from hair; and, (iv) the effects of extracted compounds such as lipids, waxes and the like on the derivatization chemistry.

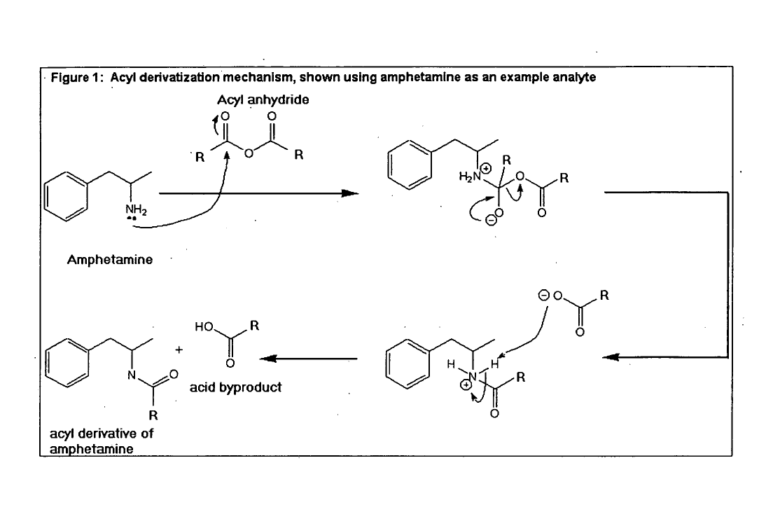

Acylation: One presently preferred approach for SFE extraction of hair involves one or more acylation reactions (e.g., see FIG. 1), wherein a drug analyte compound having one or more reactive hydrogen groups, e.g. amines, phenols or alcohols, is converted to a more stable drug-acyl derivative via nucleophilic attack at the hydrogens. The latter nucleophilic attack is mediated by an acyl donor, e.g., an anhydride, an acyl halide, an activated acyl amide such as an acylimidazole or bis(acylamide) and the like. An illustrative chemical acylation derivatization reaction is depicted in FIG. 1 wherein amphetamine is derivatized via nucleophilic attack at an amine group with acyl anhydride.

FIG. 1 depicts the steps in a chemical reaction mechanism wherein acyl addition at reactive hydrogen atoms in amphetamine results in both improved extractability and simultaneous derivatization in a supercritical CO₂ fluid. In this example, acyl addition is effected by acyl anhydride with generation of an amphetamine-N-acyl derivative product and an acid byproduct.

Often derivatization of drug analytes with acyl donors such as acyl anhydrides and acyl halides will yield acidic byproducts which, if necessary, may be removed post-extraction by evaporation under a gentle stream of nitrogen. Alternatively these may be removed from SFE, e.g. by dynamic flow of supercritical fluid through the extraction chamber. The acidic byproducts may, if necessary, be removed from the extract stream by venting a portion of the supercritical fluid, i.e., to evaporate the byproduct. For instance, when derivatization is conducted in a static mode a small portion of the supercritical fluid containing the byproduct may be vented to the atmosphere prior to collecting and trapping the extract for detection of drug or chemical analytes, e.g., see EXAMPLE 14, Method #3, below. Conditions for differential evaporation, (e.g., using a heated restrictor or evaporation chamber to control venting), are also envisaged wherein byproducts may be removed from the supercritical fluid stream while retaining the drug analyte in solution in the supercritical fluid.

In other embodiments, derivatization of drug analytes with acylimidazoles, (in contrast to acyl anhydrides and halides), produces byproducts that are relatively inert and non-acidic, i.e., imidazole. Guidance is hereby given that certain acylation reactions may give rise to derivatives with increased molecular mass. For certain detection methodologies, e.g., GC and GC/MS this may lead to undesirable decreased volatility, in this case, relative to the un-derivatized species. Therefore, the presently most preferred acylation reagents (at present) for GC and GC/MS detection are halogenated acylation reagents, and most particularly, fluorinated acylation reagents. The latter fluorinated reagents have high chemical reactivity and produce desirable drug analyte derivatives, i.e., having greater volatility and/or stability than their corresponding non-halogenated drug analytes. Fluorinated derivatives also have increased binding to certain solid phase surfaces, (e.g., treated celluloses like sulfopropyl-cellulose), which facilitates solid phase (filter) trapping from SF extracts and also facilitates their use in solid phase immunoassay protocols. Representative examples of the subject halogenated acylation reagents include, commercially available perfluoro acid anhydrides (TABLE 2), i.e., trifluoroacetic acid anhydride (TFAA), pentafluoropropionic acid anhydride (PFPA), heptafluorobutyric acid anhydride (HFBA); perfluoroimidazoles (TABLE 3), i.e., trifluoroacetylimidazole (TFAI), pentafluoropropionylimidazole (PFPI), heptafluorobytyrylimidazole (HFBD); and, N-methyl-bis[trifluoroacetamide](TABLE 4), i.e., N-methyl-bis-(trifluoroacetamide) (MBTFA). Advantageously, the most preferred halogenated fluorinated reagents are relatively volatile, allowing their possible removal (where necessary) by evaporation, or differential evaporation, from the SF stream.

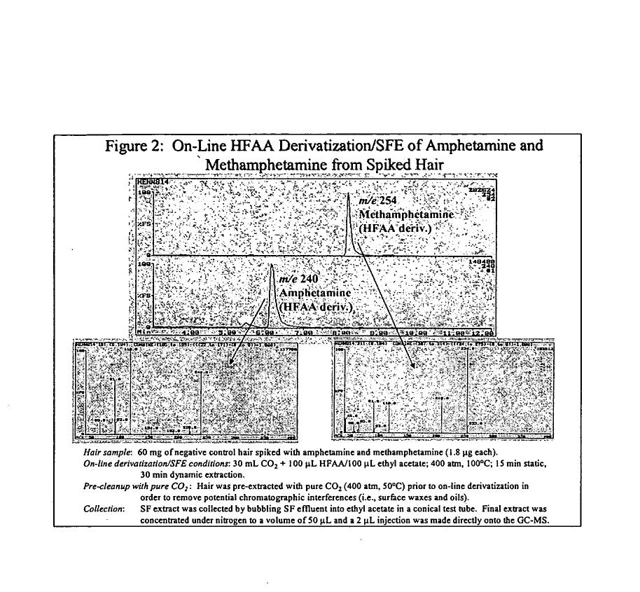

Acylation Additives: It envisaged that under certain condition it may prove desirable to add small amounts of amine bases such as pyridine or TEA to the instant acylation reaction mixtures, i.e., as catalysts, to drive the subject reactions of certain drug analytes to completion and/or to react with acidic byproducts. Preferably, any excess additive base catalysts are removed (e.g., by evaporation see EXAMPLE 14, below) prior to trapping and in-line or off-line detection of drug analyte-derivatives. For example, with detection using GC/MS residual base catalysts may prove problematic. These catalysts may also interfere with efficient downstream trapping of drug and chemical analytes from the supercritical fluid flow. In practice, catalyst additives have proven to be optional and, most fortunately, it has not proven necessary to use these reagents when acylation reactions are carried out in SF CO₂ under the instant conditions of pressure, temperature and volume (e.g., see FIG. 2).

FIG. 2 is a digital tracing depicting both a gas chromatographic separation (upper panel) and mass spectrometric analysis (lower panel) of the molecular derivative ions produced following simultaneous extraction and acyl derivatization of methamphetimine and amphetamine drug analytes in a supercritical CO₂ fluid. Hair sample: 60 mg of a negative control hair was spiked by soaking with amphetamine and methamphetimine, 1.8 μg of each. Removal of interfering substances: Background and interfering substances, i.e., hair oils and waxes, were removed by flushing the hair sample with pure CO₂ (at 50° C. and 400 atmospheres, atm, i.e., about 40.5 MPa) prior to extraction. On-line derivatization and SFE: Simultaneous derivatization and SFE were accomplished in 30 mL CO₂ (at 100 C and 400 atmospheres) to which was added 100 μL of HFAA (i.e., 0.003 v/v) and 100 μl of ethyl acetate (i.e., 0.003 v/v). Conditions for extraction were 15 min. static followed by 30 min. dynamic extraction. Collection: Extracted drug- analyte-derivative was collected by bubbling the SFE into ethyl acetate in a conical centrifuge tube. Concentration: The collected drug-analyte-derivative extract was concentrated under nitrogen to a final volume of 50 μL. GC/MS: 2 μL of the collected, concentrated drug-analyte-derivative extract were subject to detection in GC/MS.

### EXAMPLE 3

### Derivatization of Drug Analytes Having a Reactive Carboxylic Acid Group

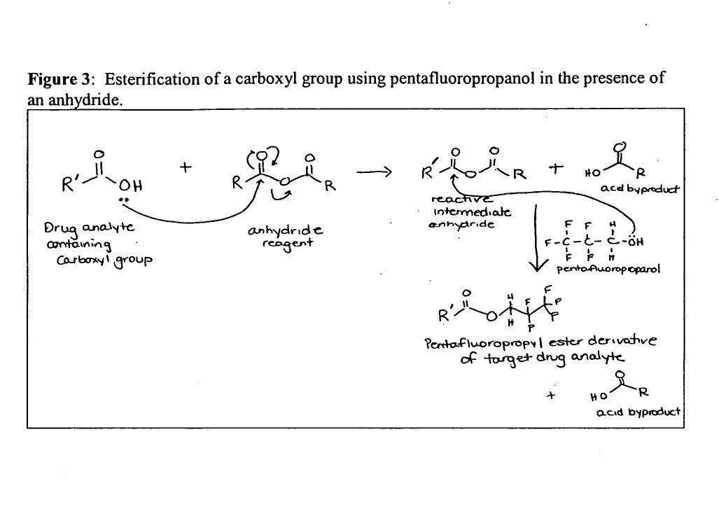

Esterification: In one presently preferred embodiment, derivatization of a drug analyte containing a —COOH group, e.g., Δ-9-THC-9-carboxylic acid or benzoylecgonine, involves esterification of the carboxyl-group by reaction with an alcohol, e.g., pentafluoropropanol (PFPOH) in the presence of an anhydride (e.g., HFBA or TFAA). The —COOH group first reacts with the perfluoro acid anhydride reagent to form an intermediate anhydride product (see e.g. FIG. 3) and because this intermediate anhydride is very reactive, it is readily attacked by PFPOH to produce the final stable pentafluoropropyl ester derivative.

Pentafluorobenzylation: Pentafluorobenzyl groups have high electron avidity in mass spectrometric methods, as well as, transient anionic stability where elimination leaves a single abundant molecular carboxylate anion. The latter process, (referred to in the GC/MS art as dissociative capture), protects the carboxyl group until application of an electron current, and then leaves an intact drug analyte or metabolite after elimination. For solid phase trapping and off-line immunoassays, the properties of high electron avidity also provide advantages of greater trapping efficiency and anionic stability in aqueous solvents.

Derivatization may be accomplished off-line prior to SFE by wetting hair samples in about 200 μl-500 μl of acetonitrile containing by volume about 18% pentafluorobenzene bromide (PFBB) and 5% pyridine. Or in-line, by introducing the PFBB/pyridine solution into the extraction chamber, followed by washing with supercritical fluid CO₂ prior to initiating SFE. Off-line, the samples are sealed e.g. in a Teflon-lined screw capped vial and heated to about 60° C. to about 80° C. for about 30 min. to about 1 hr. After cooling to room temperature the solution is brought to dryness under a stream of nitrogen, or by vacuum centrifugation, and is ready thereafter for SFE. In-line in the absence of supercritical fluid CO₂, the same temperature and pressure are effective to effect derivatization and commencing dynamic flow of CO₂ is effective to remove pyridine and reactants from the chamber (e.g., see EXAMPLE 14, below).

Simultaneous Esterification and Acetylation: Advantageously, the method set forth above, also serves to derivatize other finctional groups present in the drug analyte, e.g., alcoholic, phenolic, or amine groups which might be present in an acidic drug analyte (or in other analytes present in the extract) are commonly acetylated by the perfluoro acid anhydride, i.e., creating a second derivatized site in the subject acid drug molecule. The instant method is also most useful where there may be more than one drug analyte present because the method allows for simultaneous esterification, i.e., at carboxyl group in a first drug analyte (supra), and acetylation at an alcohol, amino or phenolic group in the second drug analyte.

### EXAMPLE 4

### Methods for Simultaneous Acyliation, Esterification and Acetylation of Drug Analytes

The chemistry of EXAMPLES 2 and 3, supra, provides basis for a highly desirable derivatization methodology utilizing combined acylation, esterification and/or acetylation reactions (supra; e.g., see FIG. 3) to provide a comprehensive extraction and derivatization method useful in testing for a variety of different drug analytes in biological specimens.

FIG. 3 depicts a chemical reaction mechanism for esterification of carboxylic acid groups in a drug analyte (R′(O)OH) with an acid anhydride results in a pentafluoropropyl-ester-drug analyte-derivative with improved extractability in a supercritical CO₂ fluid.

For example in hair drug testing, the instant methods provide the conditions necessary for the simultaneous extraction and derivatization of all of the target drugs-of-abuse set forth in TABLE 1: namely, methamphetimine, amphetamine, MDMA, MDA, cocaine, benzyoylecgonine, cocaethylene, heroin, morphine, codeine, 6-MAM, delta-9-THC, delta-9-THC-9-carboxylic acid, dihydroxyl THC, PCP, Nicotine and cotinine.

In a presently preferred embodiment, the (simultaneous or sequential) addition of a perfluoro-anhydride and a perfluoro-alcohol to an SF extraction fluid results in both improved SF extraction and simultaneous derivatization, i.e., for the desired functional groups in the target drug analyte molecules (i.e., hydroxyl, phenolic, amino, carboxyl).

### EXAMPLE 5

### Supercritical Fluid Wash, Addition and Extraction Conditions

Preferably, the instant methods employ SF-CO₂ as the primary wash and extractant in all cases, i.e., EXAMPLES 1-3, supra. Most preferably, biological samples such as hair samples are first washed with one or more super-critical fluids to remove surface background and interfering substances, e.g., oils, waxes, cosmetic detergents and skin and hair treatments and the like, as well as, possible environmental drug analyte contaminants in the form of drug particles or vapors. The latter exogenous environmental drug analytes are commonly weakly adherent to biological surfaces like hair, and pure liquid CO₂ is usually sufficient to remove them, even under subcritical conditions, e.g., see EXAMPLE 14, below. Following decontamination and removal of environmentally associated analytes, derivatization reagents according to EXAMPLES 1-4, supra, are added as follows: namely, an additive reagent comprising one or more derivatization reagents (EXAMPLES 2-4) along with any catalysts (e.g., pyridine EXAMPLE 3) and any optional reaction solvents; and any a modifier reagent (as disclosed further below). The latter modifier and additive reagents are commonly introduced directly into the supercritical fluid in the extraction chamber, i.e., in small volumes/percentages to achieve a stoichiometric excess in the intended chemical reactions. The addition to the extraction chamber may involve either manual introduction of the reagents into the chamber, e.g., opening the chamber and pipetting solutions into the chamber; or, introduction of an equipment packet containing the reagents into the extraction chamber; or, introduction of the reagents into the supercritical fluid flow, i.e., without opening the extraction chamber.

Static Extraction: The instant methods utilize first a static extraction step and then a dynamic extraction step. The static extraction is performed in a manner effective to accomplish both extraction of endogenous drug analyte (defined supra) and derivatization of that analyte. While not wishing to be tied to any particular mechanism of action, it is believe likely that derivatization of drug analytes occurs both prior to and after extraction, i.e., derivatization while the analyte is bound to the hair matrix, as well as, derivatization in the fluid phase. Thus, during the static extraction step greater than about 60% to about 80% of the drug-analyte-derivatives are formed, i.e., dependent upon the particular drug analyte. The derivatization of functional groups containing reactive hydrogens (e.g., amine, hydroxyl, carboxyl, phenolic) may proceed more rapidly than the less polar, less reactive drug analytes, and this may vary dependent upon the extractability of a particular drug-analyte or drug-analyte-derivative. Skilled artisans will of course recognize that extractions may be monitored in a kinetic manner, and as a result, time and conditions may be modified without departing from the spirit and scope of the invention.

Dynamic Extraction: Dynamic extraction is conducted by flowing a supercritical fluid and/or vapors across the surface of and around a hair sample in the extraction chamber. Physical conditions of flow rate, pressure and temperature, concentration of modifiers and/or additives and the like may be held constant, or varied in a linear or isocratic manner, during the process to achieve either removal of, or extraction of, one or more environmentally associated drug analytes. Additional disclosure follows in the EXAMPLES below.

On-line Closed-Loop Detection: It will be recognized by those skilled in the art that extraction is a dynamic process. Differential rates of extraction of drug-analytes and drug-analyte-derivatives may be used to kinetically collect extract samples for subsequent in-line or off-line analysis. Dynamic removal of sample aliquots is accomplished e.g. using a pressurized and temperature controlled tubular trapping column such as that disclosed by Stone et al. (Stone, M. A.; Taylor, L. T. 2001. Quantitative coupling of supercritical fluid extraction and high-performance liquid chromatography by means of a coated open-tubular interface. J.Chromatogr.A 931: 53-65).

### EXAMPLE 6

Extraction of Environmentally Associated Analytes, Extraction of Non-Polar Analytes Followed and/or Simultaneous Derivatization and Extraction of Polar Analytes: Acylation and Esterification

Materials and Methods follow, below, at the end of the Examples section.

According to the invention, no off-line pre-treatment or manipulation of a hair sample is absolutely required prior to its introduction into the instant extraction methods. Accordingly, pre-rinsing the hair sample, grinding, pulverization or powdering are generally not required prior to the instant SFE-based extraction methods.

Step 1. SF Decontamination of Hair Surface: Hair may be washed with optional subcritical/low supercritical CO₂ followed by supercritical SF-CO₂. Dependent on the particular conditions of the hair sample, subcritical CO₂ may remove surface contaminants, i.e., oils, waxes and environmental contaminants which might interfere or contribute background in the assay. Supercritical CO₂ under the conditions indicated is able to remove drug particles or vapors acquired through environmental exposure and thus weakly adhering to hair surface. Conditions for the optional subcritical and supercritical washes Step-#l are set forth in TABLE 1.

### TABLE 1

Removal of Interference, Background and
Environmental Analytes from Hair Samples

| | | |
|---|---|---|
| Physical Variable | Preferred Range | Most Preferred |

Subcritical/Low Supercritical Conditions

| | | |
|---|---|---|
| Pressure | 19-99 atm | 78-99 atm |
| | (2-10 MPa) | (8-10 MPa) |
| Temperature | 25-35° C. | 25-35° C. |
| Wash time: | | |
| Dynamic: | 5-30 min | 5-15 min |
| Dynamic Flow Rate: | 0.3-5 mL/min | 1-2 mL/min |
| Total Vessel Volume | 1-10 mL | 1-5 mL |
| Optional Additive(s): | H₂O 1-5% (v/v) | None |
| Derivatizing Reagent: | None | None |

Supercritical Conditions

| | | |
|---|---|---|
| Pressure | 100-450 atm | 400 atm |
| | (10-46 MPa) | |
| Temperature | 40-150° C. | 40-50° C. |
| Wash time: | | |
| Static: | 3-30 min. | 5-15 min. |
| Dynamic (flowing): | 5-60 min. | 5-15 min. |
| Dynamic SF Flow Rate* | 0.3-5 mL/min | 1-2 mL/min. |
| Total Vessel Volume | 1-10 mL | 1-5 mL |
| Optional Additive(s) | None | None |
| Derivatizing Reagent(s) | None | None |

*Dynamic SF Flow Rate, non-expanded SF.

Those skilled in the art will, of course, realized that the total dynamic wash volume and dynamic fluid flow rates will vary somewhat dependent upon the size of the SFE inlet port, the total internal vessel volume, as well as, any restrictor orifice at the outlet port from the vessel. Thus, in alternative embodiments it is anticipated that certain methods will employ vessels with larger (or smaller) internal volumes and that the fluid wash volumes and flow rates may be adjusted to achieve wash results similar to those disclosed in the wash method of TABLE 1.

The SF wash of Step 1 may contain, in addition to possible biological substances which can constitute interfering substances in certain drug detection assay formats, also purposeful adulterants added by those individuals attempting to escape detection. The latter SF wash may also contain drug particles and volatile drug materials environmentally associated with hair. Thus, provision is made in alternative embodiments for trapping and/or venting the SF wash fluid, i.e., as a waste. Provision is also made for trapping and detecting the presence or amount of one or more adulterants or environmentally associated drug analytes or chemical analytes in the supercritical fluid wash of Step 1.

Step 1A. Identification of Adulterants: In alternative embodiments, adulterants may be collected, e.g. by trapping the SF wash fluid or vapor into a solution, e.g., a solvent or buffer, or onto or through a solid phase, e.g., a C₁₈ filter or glass or steel beads. The trapped and collected adulterant and/or natural biological materials may be extracted off-line e.g., into an organic solvent, or aliquots may be take from the collected solution in the trap. In either case, the materials so-collected may be subject to assaying for the presence or amount of any possible natural biological materials, e.g., materials such as waxes and the like that are normally present and whose presence is confirmatory of a “normal” sample, as well as, for the presence of adulterants. Assays that may prove useful in such confirmation and adulterant-identification include GC/MS, immunoassays and the like as set forth supra. Thus, in this particular alternative embodiment the invention provides methods for performing a confirming assay and/or a validating assay on a test sample during the course of washing the test sample to prepare it for detection of a metabolically associated drug substance, i.e., advantageously, it is not necessary to subject two separate test samples to analysis.

Step 1B. Static Identification of Environmental Contaminants: In other alternative embodiments, it may be an aim to detect environmentally associated drug and chemical analytes. In such situations, pre-determined volumes of SF fluids and/or vapors may be collected into traps and those samples may be tested for the presence or amount of a drug or chemical analyte. Thus, in this particular alternative embodiment the invention provides methods for performing an assay for environmentally associated drug and chemical analytes, vapors and the like on a hair test sample during the course of washing the test sample to prepare it for detection of a metabolically associated drug substance. Advantageously, it is not necessary to subject the hair test sample to two separate extraction regimens or to split the sample to conduct the two different tests.

Step IC. Dynamic Identification of Environmental Contaminants: In other alternative embodiments, it may be an aim to detect the rate of elution of environmentally associated drug substances, i.e., as an additional measure of the level or extent of purposeful or inadvertent exposure, or as an additional confirming step that the materials so-associated are environmentally, and not metabolically, associated with the test sample. In this case, provision is made for periodic timed collection of SF wash fluid into a trap, i.e., trapping in a kinetic manner to determine the rate of elution of analytes from the hair test sample.

Step 1D. Real-Time Dynamic Identification of Environmental Contaminants, Adulterants and Confirming Biological Substances: In yet other embodiments, it may an aim to detect the rate of elution and/or a quantity of adulterants, or of natural confirming biological substances, or of environmentally associated drug substances in an SF wash fluid or vapor, i.e., a “real time” assay conducted on the wash fluid as it exits the SFE vessel. Provision is made, according to derivatization methods disclosed below, for conducting such an assay on an SF wash fluid. For example, the extract may be introduced in-line through one or more guard filters and/or water removal cartridges, as needed, directly into an injector for GC/MS analysis.

Step 2. On-Line Derivatization/SFE: Acylation/Esterification Approach.

Following static and dynamic washing (Step 1, supra), environmentally associated drug analytes, the test sample is decontaminated and ready for extraction of any possible metabolically incorporated drug analytes and metabolites. In different alternative embodiments provision is made for either (i) a presently preferred, extraction of metabolically incorporated drug analytes (Step 2A), or (ii) a presently most preferred, simultaneous extraction and derivatization of metabolically incorporated drug analytes (Step 2B).

Step 2A. SF Extraction: After washing (Step 1, supra) extraction of non-polar and weakly polar metabolically incorporated drug analytes is carried out by changing the SF conditions, in this case, to conditions as set forth in TABLE 2.

TABLE 2
Conditions for SFE

| Condition | Preferred Range | Most Preferred |
|---|---|---|
| Pressure: | 100-450 atm | 300-400 atm |
| | | (30.4-40.5 MPa) |
| Temperature: | 6-150° C. | 90-100° C. |
| Wash time: | | |
| Static: | 3-60 min. | 5-15 min. |
| Dynamic (flowing): | 5-60 min. | 5-15 min. |
| Dynamic SF Flow Rate* | 0.3-5 mL/min | 1-2 mL/min. |
| Total Vessel Volume: | 1-10 mL | 1-5 mL |
| Modifier: | None | None |
| Optional Additive(s): | 2-5 mM DTT | 5 mM DTT |
| | 2-5 mM cysteine | 1% TEA |
| | 0.5-1.2% (v/v) TEA | |
| Derivatizing Reagent(s): | see EXAMPLE 7 | see EXAMPLE 7 |

*Dynamic SF Flow Rate, non-expanded SF.

Step 2B. Simultaneous SF Extraction and Derivatization: After washing (Step 1, supra) extraction is carried out by changing the SF conditions, i.e. with modifier and derivatizing agent. Addition of the latter agents may be off-line, e.g., by cooling the extraction chamber, opening it and adding the modifier and derivatization reagents, but preferably the latter agents are either (i) introduced directly into the SF fluid flowing into the extraction vessel containing the test sample, or (ii) introduced into the extraction chamber in an equipment packet with the original hair sample, i.e., as set forth further below in EXAMPLE 14-15. Simultaneous extraction, acylation and esterification of drug and chemical analytes is preferably conducted as a static extraction step. The subject derivatizing agents and additives accomplish SF extraction involving possible breakage of covalent bonds and/or electrostatic displacement, but also with concomitant acylation of amine, hydroxyl, and phenolic groups and/or esterification of carboxyl groups. Advantageously, the derivatives formed in this manner are less polar, less reactive, and more readily extractable analogs that have a lower Ka for electrostatic, i.e., non-specific, re-binding to the hair matrix.

TABLE 3
Static Conditions for Simultaneous SFE
and Acylation and Esterification

| Physical Variable | Preferred Range | Most Preferred |
|---|---|---|
| Pressure | 100-450 atm | 300-450 atm |
| | | (30.4-45.6 MPa) |
| Temperature | 6-150° C. | 80-110° C. |
| Wash time: | | |
| Static: | 3-60 min. | 5-15 min. |
| Dynamic (flowing): | 5-60 min. | 5-15 min. |
| Dynamic SF Flow Rate* | 0.3-5 mL/min | 1 mL/min. |
| Total Vessel Volume | 1-10 mL | 1-5 mL |
| Modifier(s) | Ethyl acetate | Ethyl acetate |
| | Methanol | |
| Derivatizing Reagent(s) | see Example 7 | see Example 7 |

*Dynamic SF Flow Rate, non-expanded SF.

The extracted drug analytes may be subject to direct detection, e.g., in-line trapping and GC/MS, or solid phase or solution trapping with subsequent off-line detection e.g. using immunoassay. For those drugs which normally require derivatization prior to GC/MS to effect detection, detection may advantageously be accomplished without the requirement for intervening manual steps, e.g. concentration and off-line derivatization. For those other drugs where derivatization is not required, the drug analytes may be detect in the same mixed extract solution. Thus, an additional advantage of the invention is simultaneous extraction of drugs requiring derivatization with those which do not, and detection of both types of analytes in the same extract mixture.

TABLES 4-7 include lists of preferred and most preferred derivatization reagents, i.e., with chemical reagent abbreviations. Preferably in sequential addition methods, the first derivative added comprises a perfluoro anhydride or alcohol, i.e., PFPOH or HFAA and anhydrides as set forth in TABLE 4, most preferably, HFAA and PFPOH.

### EXAMPLE 7

### Extraction of Non-Polar Analytes followed and/or Simultaneous Derivatization and Extraction of Polar Analytes: Methylation, Alkylation, Silylation

In Situ Static Derivatization:

(a) Methylation and Alkylation: Polarity of chemical analytes, pesticides, steroids, drug analytes, and their respective metabolites containing hydroxyl-, carboxyl-, phenyl-, amide- and amine-groups may be decreased by conversion to methyl and alkyl esters and ethers. While this normally occurs slowly under SFE conditions in the presence of methanol modifiers, this process is accelerated through the use of catalytic amounts of HCl in water, formic acid, alumina and cations.

(i) As a first alternative, following decontamination of the hair surface (Step 1, EXAMPLE 6 above) CO₂, methanol and catalytic amounts of HCl (e.g., IN HCl in distilled water), in proportions 80:20:1, respectively, are introduced into the extraction vessel. To promote derivatization in the presence of catalyst, the vessel temperature and pressures are maintained at about 70° C. to 90° C. and about 175 atm (17.7 MPa) to 210 atm (21.2 MPa) for about 20min. to about 3 hrs., preferably about 10 min to about 20 min, with no flow, i.e., prior to commencing dynamic extraction.

(ii) As a second alternative, under the same conditions of temperature, pressure and flow as in (i) supra, catalytic amounts of H⁺ or other cations ions may be added to the sample extraction chamber in the form of a solid phase cation exchanger pre-equilibrated e.g. in IN HCl and then encapsulated and/or coated with a methanol-soluble protectant, i.e., the protected-exchanger is added with the hair sample to the extraction chamber which is preferably quartz- or glass-lined stainless steel to minimize corrosion. Following decontamination of the hair surface in CO₂, the addition of methanol modifier liberates the catalytic cation exchanger from the methanol-soluble protectant and derivatization proceeds. Representative examples of the instant methanol-soluble protectants include e.g. methanol soluble plant and animal oils, waxes and lipids, with the requirement that the instant protectants are not freely solubilize in supercritical CO₂ under the conditions of extraction, but are freely soluble in methanol-modified CO₂.

(iii) As a third alternative, under the same conditions of temperature, pressure and flow as set forth in (i) supra, neat alkyl halides such as methyl iodide or parafluorobenzyl bromide(PFBBr) are used, i.e., instead of methanol, as the reagent in base catalyzed alkylation. The subject reaction with methyl iodide may be accomplished in supercritical CO₂ with catalytic amounts of organic or inorganic bases, anion-exchange resins or triethylamine additive. SFE alkylation with PFBBr may be promoted in the presence of catalytic amounts of potassium iodide or triethylamine. (iv) As a fourth alternative, trimethylphenylammonium hydroxide acts both as a modifier, i.e., ion pair donor, and as a methylating agent. In alternative embodiments, the instant methylation reaction may also be conducted as a wet chemical pretreatment of hair e.g. using 0.5M methyl iodide in acetonitrile.

(b) Acetylation: Acetylation is also useful for reducing polarity of phenolic drug analytes and their metabolites. In situ acetylation is accomplished using supercritical CO₂ containing e.g., an acetylation reagent such as acetic anhydride and a solvent additive such as triethylamine, pyridine or SAX and water. The latter components may e.g. be pumped sequentially into the extraction chamber, or the acetylation reagent may be contained in a CO₂ insoluble form in the chamber and liberated upon addition of the triethylamine.

(c) Silylation: Silylation is useful for reducing the polarity of analytes containing carboxyl-, hydroxyl- and phenyl- groups.

Dynamic Derivatization:

Following decontamination of the hair surface (Step 1 EXAMPLE 6 above), under the extraction conditions set forth in EXAMPLE 8, supra, derivatizing agents are introduced into the extraction chamber, e.g., (a) directly into the dynamic flowing SF stream, and/or (b) through an injection port fitted e.g. with a metering valve and/or an injection pump. The amount of derivative introduced in this manner will of course vary dependent upon the test parameters e.g. (i) the type and amount of drug analyte constituting a positive determination of drug usage, (ii) the amount and type of hair sample being evaluated, (iii) the level of detection required to effect a positive identification of drug usage in light of the common background levels encountered and the like. Those of ordinary skill in the art of testing for drugs of abuse will, of course, recognize that the quantity of added derivative(s), i.e., as measured in volume and molar amounts, may vary slightly upon the latter test parameters, and that adjustments may be made to achieve the desired result: namely, overcoming background, possible adulteration and environmental contamination. Preferably, the subject derivatization reagent is added to the hair sample in stoichiometric excess over any expected drug analyte to be detected. It is also anticipated that multiple different derivatives may be added either simultaneously, or sequentially.

### EXAMPLE 8

### Extracted Derivatized Analytes: Dynamic Extraction and Collection Modifiers and Additives

Supercritical fluid effluent from EXAMPLE 7 contains possible perfluoroacyl-, alkyl-, methyl-, acetyl- and silyl-derivatives and the like of drug and chemical analytes, as well as, possible reaction byproducts e.g. perfluoroacids of perfluoroacyl-derivatives.

Dynamic Modifiers and Additives: Derivatized analytes, while generally soluble in supercritical CO₂, may have their extractability increased through the continuous prudent use of compatible solvent systems such as modifiers in the amount of about 1% v/v to about 20% v/v of the dynamic flow CO₂ volume. The following solvent modifier systems are presently preferred: namely, (a) methanol; (b) methanol containing about 0.5% (v/v) to about 1.2% (v/v) TEA as additive; (c) methanol containing about 0.5% (v/v) to about 1.2% (v/v) DEA as additive; (d) ethanol containing about 0.5% (v/v) to about 1.2% (v/v) TEA additive; (e) ethanol containing about 0.5% (v/v) to about 1.2% (v/v) of DEA additive; (f) propanol; and, (g) mixtures of dichloromethane, dichloroethane, methanol, acetone, butylacetate and water as set forth further below, and in EXAMPLE 14, below. Highly basic primary amines are preferably not used as additives in supercritical CO₂ since they can form carbamates.

Alternative presently preferred solvent modifier systems for acetylated derivatives include the following: namely, (a) 1,2-dichloroethane (DCE):methanol:water at a ratio of about 88 to 89 parts DCE to about 11 to about 12 parts methanol and about 0.1 part water (e.g., 88:11:0.1, 89:12:0.1 and the like); (b) DCE:acetone:water (50:50:1); and (c) butylacetate:acetone (7:3).

Alternative presently preferred solvent modifier systems for methylated derivatives include the following: namely, (a) methanol containing about 0.02% v/v water; (b) ethylacetate:pyridine:water (12:5:4); and, (c) n-propanol:1N NH₄OH: water (6:2:1).

### EXAMPLE 9

### Enhanced Drug Extraction: Glycoconjugate Modification and Fragmentation for SFE

Supercritical fluid extraction of phenolic compounds, drug analytes, pesticides, steroids, their respective metabolites and the like is enhanced after fragmentation of hair-matrix complex carbohydrates, i.e., in blood group glycosphingolipids and glycoproteins. The fragmentation is accomplished using one or more wet chemical pretreatment steps, or alternatively, is accomplished in situ during static or dynamic SFE extraction. The respective wet chemical pretreatment methods are disclosed first, followed by in situ SFE methods.

Sulfoglycolipids, sulfatides, chondroitin sulfates, steroid sulfates: (i) As a first alternative embodiment, sulfate containing compounds in hair are cleaved by solvolysis with acidified tetrahydrofuran for 1 hr. at 50° C., the samples are taken to dryness under a stream of nitrogen and the hair samples prepared for SFE as described in the examples above. (ii) As a second alternative embodiment, extraction is conducted using a methanol modifier containing about 0.2M to about 0.5M of an additive capable of both providing an ion pair to improve extraction during SFE and functioning as a derivatizing agent under conditions of later GC separation, e.g., forming alkyl esters in the injection port of the GC. Representative example of the subject modifiers are provided by tetrabutylammonium hydrogen sulfate, tetraethylammonium hydrogen sulfate, tetramethylammonium hydrogen sulfate, trimethylphenylammonium hydroxide and (trifluoromethyl)phenylammonium hydroxide.

Derivatization of hair glycoconjuzates: Sugar hydroxyl groups contribute electrostatic charge for drug-analyte binding. Derivatization of the hair matrix constitutes one method for decreasing these associative binding interactions between a drug analyte or metabolite and the hair matrix. Methods described in EXAMPLE 7, “Static Derivatization” supra, for methylation in methanolic-HCL, cation-modified methanol and acetylation, e.g. with acetic anhydride, are also useful for altering the physical properties of glycoconjugates in the hair matrix and thereby decreasing drug binding in those matrices. Derivatization of glycoconjugates in the hair matrix may also be accomplished in a wet chemical procedure, i.e., in methanolic HCl, 60° C. for 1-3 hrs. prior to SFE extraction.

Fragmentation of hair glcoconjugates: Acid hydrolysis: Prior to commencing SFE, hydrolysis of hair glycoconjugate glycolipids and glycoproteins is accomplished as a static treatment prior to initiating dynamic SFE. The hydrolysis is accomplished using weak acids or methanolic acids or bases, in situ (in the extraction chamber), in a small volume at a density of CO₂ having good solvating power, i.e., conditions as follows: namely, (i) hydrolysis in with about 10 μl to about 200 μl volume of the hydrolysis acid or base, preferably about 50 μl to about 100 μl; (ii) at a temperature of about 35-115 C, preferably about 40° C. to about 80° C. and most preferably about 40° C. to about 60° C.; and, (iii) for an incubation time of about 10 min. to about 2 hrs., preferably about 10 minutes to about 45 minutes, most preferably about 10 minutes to about 20 minutes. Representative weak acids so useful in the instant method include phosphoric acid at a concentration of about 10% v/v to about 20% v/v, preferably about 16.5% v/v; phthalic acid at a concentration of about 0.07M to about 0.1 2M, preferably about 0.1M; and, formic acid at a concentration of about 30% to about 70%. Representative methanolic acids include 1N HCl in methanol. Representative methanolic bases include 1N NaOH in methanol.

Fragmentation of hair glycoconjugates: Enzymatic hydrolysis: Treatment with one or more glycosidases and/or endoglycosidase is accomplished in an aqueous buffer at about 35° C. to about 38° C., either off-line, i.e., prior to the hair sample being placed into the extraction chamber, or in-line in a static mode in aqueous buffer prior to supercritical fluid extraction. Representative glycosidases useful for fragmentation of hair glycoconjugates include hexosaminidases, endoglycosidase H and I, sialidases, galactoaminidases, glucosaminidases, glucosidases, galactosidases, phytases, chitinases and the like.

### EXAMPLE 10

### Extraction Conditions

The physical properties of supercritical CO₂ change with density and are dependent upon at least pressure and temperature. Solvating power and extraction volume relate to density. For example, in theory, at constant temperature as pressure decreases (i) density decreases, (ii) solvating power decreases, (iii) extraction time may increase, (iv) dynamic elution volume in which extracted analytes are contained may increase, but advantageously, (v) diffuisivity of solvent into hair may increase. At constant pressure, as temperature increases (i) density decreases, (ii) solvating power of CO₂ decreases, (iii) vapor pressure of any modifiers and/or additives increases, but somewhat paradoxically, (iv) extractability of certain analytes may increase. Thus, in practice, solvating power and diffuisivity as determined by temperature and pressure constraints need to be matched with the hair matrix properties that bond drug and chemical analytes in hair. However, if extraction volume is large the dilute solution of drug- and chemical-analytes extracted at high temperature and low pressure may prove difficult to capture and detect; and chemical stability of the analytes also becomes problematic. In the art, factoral experimental designs have been used in an attempt to approach the multivariate analysis needed to optimize analyte extraction, e.g., including at least the following factors: namely, sample pre-treatment condition(s), pressure, temperature, modifier volume, additive volume, static extraction time/conditions, dynamic extraction time/conditions, extraction volumes, solid (or fluid phase) collection conditions and the like. In view of the unknown properties of chemical and drug analyte interactions with the hair matrix, proteins and blood group glycolipids, optimal SFE conditions for simultaneous extraction of polar and non-polar drug- and chemical-analytes were not believed to be obvious.

Physical interactions between the hair matrix glycoconjugates and drug- and chemical-analytes can be disrupted by primarily by changing the ionic charge of glycosyl atoms including oxygen (hydroxyl), sulfur (thiol, SO₂, SO₃), nitrogen (amide, amine) and/or by changing solvent conditions in a manner that effectively remove waters of hydration needed to stabilizing binding interactions.

Additive Mixtures: Amines, PEG and buffer at pH 8: The pKa values for sugar ring hydroxyl residues vary, but basic pH (pH>8.5) favors deprotonation. Supercritical CO₂ exhibits pH properties of carbonic acid, e.g., pH 2-3. Amines additives (supra) such as diethyl- and tri-ethylamine lack substantial buffering capacity. Buffering to >pH 8 is achieved using organic buffers in an aqueous solution, e.g. Tris-base at a final concentration in the extraction fluid of about 0.1 mM to about 10 mM, preferably about 1 mM to about 5 mM and most preferably about 1 mM to about 2.5 mM. Waters of hydration are removable by poly-ethylene-glycols, i.e., about 1% (w/v) to about 15% (w/v) of PEG-1500 in the extraction fluid, preferably about 3% to about 8% and most preferably about 4% to about 6%.

Zwitterion Additives: Zwitterion additive mixtures possess chemical compounds which simultaneously possess positive and negative charge and preferably also contain hydrophobic properties. Commonly employed as detergents, in contrast, the instant use herein is as ionic species with hydrophobic properties, i.e., commonly each detergent, or combination of detergents, are used at concentrations well below their critical micelle points. The subject zwitterions compounds are preferably formulated alone, or in combination mixtures, so that they are present in the modified supercritical CO₂ extract solution at a final (combined) concentration of about 0.1% (w/v) to about 5% (w/v), preferably about 0.1% (w/v) to about 2.5% (w/v) and most preferably, about 0.1% (w/v) to about 1.5% (w/v). In the case of mixtures of zwitterionic compounds, preferably, in the final modified supercritical CO₂ extract solution each zwitterionic compound is present in about equal molar amounts.

Representative examples of the subject zwitterionic compounds include amino acids such as Leucine, Isoleucine, Valine, polymeric forms thereof and the like; glycolipid gangliosides containing a sialic acid and an amino sugar; and, zwitterionic detergents such as 3-[(3-chloramidopropyl)dimethylammonio]-2-hydroxy-1-propanesulfonate (CHAPSO); 3-[(3-chloramidopropyl)dimethylammonio]-1-propanesulfonate (CHAPS); N-decyl-N,N-dimethyl-3-ammonio-1-propane-sulfonate (NDAPS); phosphatidyl-choline dipalmitoyl and the like. When compressed and decompressing the subject additive compound mixtures foaming may result. Guidance is given as follows: To control foaming, and add additional zwitterionic potential, silicone- and non-silicone based antifoaming agents are optionally added to the subject Zwitterion Additive mixture in an amount that is equal to about 1.0% (v/v) to about 20% (v/v), preferably about 5% (v/v) to about 20% (v/v) and most preferably about 10% (v/v) to about 20% (v/v). Representative examples of the subject antifoaming agents include polyols such as Antifoam 204; mixtures of silicones and polyols such as Antifoam 289; silicones such as Antifoam A and Antifoam B (e.g., Sigma Chemicals, St. Louis, Mo.). As an alternative to antifoaming agent, diatomaceous earth, e.g., Celite 566, Hydromatrix (Varian, Harbor City, Calif.) is added to the sample chamber with the hair sample in an amount at least equal to 10-times the weight of hair. Preferably, after addition of hair a machine packet filled with diatomaceous earth is inserted into the extraction chamber.

Guidance is also given that in certain aqueous solutions the subject detergents may be waxy and relatively insoluble, however they may be relatively soluble in alcohols and organic solvents. Thus, it may prove desirable to dissolve and/or suspend the subject additive compounds directly in modifier solutions rather than attempting to add them to the supercritical fluid with aqueous additive mixtures.

Representative conditions for extraction are as follows: namely, (i) a pressure of about 100 bar to about 400 bar, preferably about 200 bar to 400 bar, most preferably about 300 bar to 400 bar; (ii) a temperature of about 35° C. to about 75° C., preferably about 40° C. to about 70° C., most preferably about 40° C. to about 60° C.; (iii) a static extraction time of about 3 minutes to about 20 minutes, preferably about 5 to about 15 minutes and most preferably about 10 minutes to about 15 minutes; and, (iv) a dynamic extraction flow rate of about 0.8 mL/min. to about 3 mL/min., preferably about 1 mL/min. to about 3mL/min., and most preferably about 1 mL/min to about 2mL/min.

Anionic/Cationic Detergent Additives: Mixtures of anionic and cationic detergent compounds are useful for contributing all of the following to an additive mixture, namely, a positive- and a negative-charged ions and hydrophobic properties. Commonly employed as detergents, in contrast, the instant use herein is as ionic species with hydrophobic properties, i.e., commonly each detergent, or combination of detergents, are used at concentrations well below their critical micelle points. The subject anionic and cationic compounds are preferably formulated in combination mixtures so that they are present in the modified supercritical CO₂ extract solution at a final (combined) concentration of about 0.1% (w/v) to about 5% (w/v), preferably about 0.1% (w/v) to about 2.5% (w/v) and most preferably, about 0.1% (w/v) to about 1.5% (w/v). Preferably, in the final modified supercritical CO₂ extract solution the total combination of different anionic and cation detergent compounds are present in a ratio of about 1:2 to about 1:4, i.e., about two-times to about four-times more final total cation than anion.

Representative examples of anionic detergents so useful include sodium salts of the following compounds: namely, alginic acid (i.e., β₁₄ -D-mannuronic acid polymers); caprylic acid; cholic acid; decane sulfonic acid; dehydrocholic acid; deoxycholic acid; dioctyl sulfosuccinate; dodecanesulfonic acid; glycocholic acid; glycodeoxycholic acid; hepatane sulfonic acid; hexane sulfonic acid; lauroylsarcosine; lauryl sulfate; nonanesulfonic acid; octanesulfonic acid; pentanesulfonic acid; taurocholic acid; taurodeoxycholic acid; Teepol HB7; Tergiton; Triton and the like. Representative examples of cationic detergents so useful include the following: namely, alkyltrimethylammonium bromides, benzalkonium chloride, benzyldimethyldodecyl ammonium bromide; benzyldimethylhexadecyl ammonium chloride; benzyldimethyltetradecyl ammonium chloride; cetyldimethylethyl ammonium bromide; cetylpyridinium bromide, cetylpyridinium chloride, decamethonium bromide, dimethyldioctadecyl ammonium bromide; methylbenzethonium chloride; methyl mixed trialkyl ammonium chloride; methyl trioctylammonium chloride; N,N′,N′-polyoxyethylene(10)-N-tallow-1,3-diamino propane and the like.

When compressed and decompressing the subject additive compound mixtures foaming may result. Guidance is given as follows: To control foaming, and add additional zwitterionic potential, silicone- and non-silicone based antifoaming agents are preferably added to the subject Zwitterion Additive mixture in an amount that is equal to about 1.0% (v/v) to about 20% (v/v), preferably about 5% (v/v) to about 20% (v/v) and most preferably about 10% (v/v) to about 20% (v/v). Representative examples of the subject antifoaming agents include polyols such as Antifoam 204; mixtures of silicones and polyols such as Antifoam 289; silicones such as Antifoam A and Antifoam B (e.g., Sigma Chemicals, St. Louis, Mo.). Guidance is given that in certain aqueous solutions the subject detergents may be waxy and relatively insoluble, however they may be relatively soluble in alcohols and organic solvents. Thus, it may prove desirable to dissolve and/or suspend the subject additive compounds directly in modifier solutions rather than attempting to add them to the supercritical fluid with aqueous additive mixtures.

Representative conditions for extraction are as follows: namely, (i) a pressure of about 100 bar to about 400 bar, preferably about 200 bar to 400 bar, most preferably about 300 bar to 400 bar; (ii) a temperature of about 35° C. to about 75° C., preferably about 40° C. to about 70° C., most preferably about 40° C. to about 60° C.; (iii) a static extraction time of about 3 minutes to about 20 minutes, preferably about 5 to about 15 minutes and most preferably about 10 minutes to about 15 minutes; and, (iv) a dynamic extraction flow rate of about 0.8 mL/min. to about 3 mL/min., preferably about ImUmin. to about 2 mL/min., and most preferably about 1 mL/min to about 2 mL/min.

### EXAMPLE 11

### Tandem Extraction: Static Extraction with Enhanced Fluidity Liquid Chromatography Followed by Dynamic SFE

Enhanced FluidityLiquid Chromatography: Supercritical fluid (SF) CO₂ is useful for lowering the viscosity and increasing the diffusivity of organic solvents into hair, and with the advantage that at about 40-60% final volume of SF CO₂ the polar properties of the organic solvent are somewhat retained. This method is useful for extracting polar drug analytes and metabolites, e.g., THC-acid, benzoylecgonine, methamphetamine, amphetamine, MDA, heroin and the like.

Optional Step #1: With Pretreatment: Hair samples may be pre-treated off-line, or in the extraction chamber, with the respective solvent systems as set forth below, i.e., in the absence of supercritical CO₂. Preferably, the subject samples are treated at a room temperature of about 20° C. to about 30° C., preferably about 25° C. for about 3 minutes to about 15 minutes, preferably about 5 minutes to about 15 minutes, most preferably about 5 minutes to about 10 minutes. The pre-treated samples are next processed according to Step #2, below. As an alternative, samples may be pre-treated according to EXAMPLE 12 below prior to initiating Step #2.

Step #2: With, or without, pretreatment according to Step #1, above, or the method of EXAMPLE 12, below, samples are next treated as follows. Namely, the required amount of the respective different organic solvents, as set forth below, are loaded into the extraction chamber with the hair sample, preferably using an equipment packet containing the requisite measured amounts of the solvent reagents, e.g., a temperature or pressure permeable packet. A measured amount of CO₂ is next metered into the extraction chamber to achieve a final amount in the chamber of about 40% (v/v) to about 60% (v/v) and the chamber is then sealed. Next, without SF CO₂ flow, the chamber is brought to pressure and temperature and held under those conditions for about 3 minutes to about 15 minutes, preferably about 5 minutes to about 15 minutes, most preferably about 5 minutes to about 10 minutes. After this time, dynamic extraction is initiated by gradually introducing SF CO₂ into the chamber at a rate of about 0.5 ml/min to about 3 mL/min, preferably about 0.5 mL/min to about 2 mL/min, most preferably about 1 mL/min to about 2 mL/min. The extract is collected for subsequent off-line assay, e.g., immunoassay, or is fed into a solid phase or liquid trap for subsequent in-line, or off-line, GC/MS analysis.

Static Solvent System: Analytes associated with complex carbohydrates are releasable in chloroform:methanol:water at ratios in the range of (70-80: 10-20: 1-5), preferably about (80:20:5), and this solvent system may be useful in Step #1. However, chloroform is not a desirable solvent for SFE, and preferably, dichloromethane (DCM) or dichloroethane (DCE) is substituted for use in Step #2, i.e., DCM:methanol:water (70-80: 10-20: 0.1-5) and/or DCE:methanol:water (70-80:10-20:0.1-5). Other useful solvent systems include the following: namely, n-propanol:water (40:10 v/v); methyl-isobutyl-ketone: methanol: water (40:80:30 v/v/v); ethylacetate: methanol: water (40:72:28 v/v/v); methylacetate:methanol:water (40:72:28 v/v/v); and, petroleum ether:isopropanol:water (40:112:38 v/v/v). Presently preferred solvent systems are n-propanol:water (40:10 v/v) and methylisobutylketone:methanol:water (40:80:30 v/v/v).

### EXAMPLE 12

### Tandem Extraction: Static Extraction With Subcritical Solvent Extraction Followed by Dynamic SFE

Subcritical Solvent Extraction: In closed systems under constant pressure, organic solvents at temperatures at or near their vapor points can exhibit properties of increased diffusivity while retaining solvation properties.

For subcritical solvent extraction, a hair sample is placed into an extraction chamber. An organic solvent system according to EXAMPLE 11, above, is added to the chamber, preferably enclosed in a reagent packet, e.g., a temperature or pressure sensitive reagent packet. Next, the chamber is sealed and while monitoring pressure the temperature is raised to about the vapor point of the most volatile component in the solvent system. Maintaining constant pressure, static extraction is allowed to continue for about 3 minutes to about 15 minutes, preferably about 5 minutes to about 15 minutes, most preferably about 5 minutes to about 10 minutes.

### EXAMPLE 13

### General Dynamic Conditions of SFE Temperature and Pressure: Derivatized and Non Derivatized in the Presence and Absence of Modifiers and Additives

The presently preferred conditions for conducting SFE of hair are set forth in TABLES 1-4 above and in TABLES A and B, below. Conditions for determining environmental exposure are set forth separately from those for determining metabolic incorporation into hair.

TABLE A
Preferred SFE Conditions*

| | | | | | | | |
|---|---|---|---|---|---|---|---|
| | | | Optional | | | | |
| | | Modifier | Additive(s) | CO₂ Flow | Temp. | Pres. | Time |
| Condition # | Analyte(s) | (% v/v) | (% v/v) | (mL/min) | (° C.) | (MPa) | (min) |

Environmental Contamination

| | | | | | | | |
|---|---|---|---|---|---|---|---|
| 1 | Non-Polar | None | None | 0.5-3 mL/min | 25° C.-35° | 2-1016 | 5-30 |
| | Drug and/or | | | | | | |
| | Chemical | | | | | | |
| 2 | Polar Drug | None | None | 0.5-3 mL/min | 25° C.-55° C. | 2-22 | 5-30 |
| | and/or | | | | | | |
| | Chemical | | | | | | |

Metabolic Incorporation

| | | | | | | | |
|---|---|---|---|---|---|---|---|
| 3 | Non-Polar | None | None | 0.5-3 mL/min | 40° C.-55° C. | 9.5-20.6 | 5-30 |
| | Drug and/or | | | | | | |
| | Chemical | | | | | | |
| 4 | Polar Drug | MeOH | TEA and/or | 0.5-3 mL/min | 40° C.-55° C. | 20.0-27.2 | 5-30 |
| | and/or | and/or | BA and/or | | | | |
| | Chemical | DCM to | H₂O to a | | | | |
| | | a final | final | | | | |
| | | 1-30% | 0.5-1.2% | | | | |
| 5 | Polar Drug | None | None | 0.5-3 mL/min | 60° C.-110° C. | 27.3-55.5 | 5-30 |
| | and/or | | | | | | |
| | Chemical | | | | | | |

*1 psi is about equal to 6895 Pa; 1 atm is about 14.7 psi (pounds per square inch of pressure); MeOH, methanol; DCM, dichloromethane; TEA, triethylamine; BA, n-butylamine; Temp., temperature; Pres., pressure; Modifier and Additive concentration are expressed as % (v/v), percentage volume of the modifier or additive in a given volume of SF CO₂.

TABLE B
Most Preferred SFE Conditions*

| | | | | | | | |
|---|---|---|---|---|---|---|---|
| | | | Optional | | | | |
| | | Modifier | Additive(s) | CO₂ Flow | Temp | Pres | Time |
| Condition # | Analyte(s) | (% v/v) | (% v/v) | (mL/min) | (° C.) | (MPa) | (min) |

Environmental Contamination

| | | | | | | | |
|---|---|---|---|---|---|---|---|
| 1 | Non-Polar | None | None | 1-2 mL/min | 25° C.-35° | 8-10 | 5-15 |
| | Drug and/or | | | | | | |
| | Chemical | | | | | | |
| 2 | Polar Drug | None | None | 1-2 mL/min | 35° C.-50° C. | 8-22 | 5-15 |
| | and/or | | | | | | |
| | Chemical | | | | | | |

Metabolic Incorporation

| | | | | | | | |
|---|---|---|---|---|---|---|---|
| 3 | Non-Polar | None | None | 1-2 mL/min | 40° C.-55° C. | 9.5-20.6 | 5-15 |
| | Drug and/or | | | | | | |
| | Chemical | | | | | | |
| 4 | Polar Drug | MeOH | TEA and/or | 1-2 mL/min | 40° C.-55° C. | 20-27.2 | 5-15 |
| | and/or | and/or | BA and/or | | | | |
| | Chemical | DCM to | H₂O to a | | | | |
| | | a final | final | | | | |
| | | 10-30% | 0.5-0.75% | | | | |
| 5 | Derivatized | None | Ethyl acetate | 1-2 mL/min | 90° C.-110° C. | 30.4-40.5 | 5-15 |
| | Polar Drug | | 0.5-0.75% | | | | |
| | and/or | | | | | | |
| | Chemical | | | | | | |
| 6 | Derivatized | MeOH | HCl - | 1-2 mL/min | 90° C.-110° C. | 30.4-40.5 | 5-15 |
| | Polar Drug | 1-2% | catalytic | | | | |
| | and/or | | amnts; | | | | |
| | Chemical | | H₂O | | | | |

### EXAMPLE 14

### Tandem In-Line Extraction of Polar and Non-Polar Analytes

Equipment:

Extraction Chamber:

Supercritical Fluid Inlet Port: Yes

Modifier/Additive Inlet Port: Yes

Inlet Pumps: Yes, as necessary for SF CO₂,

Inlet Mixing Chamber: Optional

Outlet Restrictor: Yes—i.e., a flow valve capable of controlling both flow rate and establishing static (no flow) conditions.

Outlet Restrictor Heating: Yes

Outlet extract collecting pumps: Optional as needed for mixing with a trap-compatible solvent system

Flow monitoring: Yes.

Trap #1:

Solid Phase Filter Cartridge suitable for solid phase immunoassay

Trap #2:

Solid phase cartridge e.g., C₁₈ column, suitable for trapping and GC/MS elution

Trap Temperature Control: Yes, i.e., −10°C. to 10° C., e.g., a recirculating water
bath or ice water bath.

Trap Pressure Control: Yes.

Reagents:

Extraction Fluid:

Supercritical CO₂

Stock Modifier Solution #1:

20-30% (v/v) Methanol in dichloromethane

Stock Modifier Solution #2:

0.5-0.7% (v/v) Water in absolute methanol

Stock Modifier Solution #3:

Dichloromethane:methanol:water at volume ratios of (80:20:0.5), respectively.

Stock Modifier Solution #4:

10-20% (v/v) water in methanol

Stock Additive Solution #1:

Triethylamine—reagent solution, e.g. 0.73 g/ml Sigma #T0886

Stock Additive Solution #2:

n-Butylamine—reagent solution, e.g. 0.74 g/ml Sigma #B2266

Stock Additive Solution #Triethylamine:butylamine (1:1) —i.e., a 1:1 (v/v) mixture of Additive

Solution #1 and #2, supra.

Stock Additive Solution #4:

Acetic anhydride—in a concentration and amount effective for derivatization according to EXAMPLE #6-#7, supra.

Illustrative Method #1:

Pumped and Metered Modifier and/or Additive Solutions:

(1) Place hair sample into the extraction chamber with both static mode and dynamic mode flow controls;

(2) After sealing the chamber, initiate flow of SF CO₂ and in using a short 3-5 minute static treatment mode bring the temperature and pressure of the chamber to a temperature and pressure effective to remove environmentally associated drug- and chemical-analytes while retaining metabolically incorporated drug analytes, e.g., a preferred or most preferred temperature and pressure as set forth in TABLE A and TABLE B, respectively, Condition #1, for non-polar analytes, and/or Condition #2, for polar analytes (EXAMPLE # 13, supra);

At the equilibrated temperature and pressure, initiate dynamic flow and wash the hair sample, i.e., at a flow rate according to Condition #1 or #2, supra, to remove the environmentally associated drug analyte(s) and chemical analyte(s) such as pesticides, herbicides, nicotine compounds and the like which may either constitute chemical analytes of interest, or alternatively, may constitute potential interfering or background substances in the detection assay;

(4) Collect the wash sample, or a portion of the wash sample, for an off-line testing procedure such as an immunoassay or GC/MS procedure;

(5) Using metered flow control and pumping, inject an amount of SF CO₂, Modifier Stock Solution #1, #2 or #3 and optional Additive Stock Solution #1, #2 or #3 into the chamber to achieve final volume of modifier and optional additive as set forth in TABLE A or TABLE B, Condition #4 (EXAMPLE 13, supra). When the requisite volume percentages have been reached, terminate the flow of supercritical fluid and switch to a static extraction mode under the different conditions of temperature and pressure effective to accomplish elution of any polar analytes incorporated into the hair matrix, e.g., Condition #4 TABLE A or TABLE B, supra (EXAMPLE 13);

(6) Switch back to a dynamic mode of extraction by resuming flow of the supercritical fluid, i.e., at the flow rates indicated in either of TABLE A or TABLE B Condition #4, to elute the extracted polar analytes and then, as modifiers and any optional additives are progressively flushed from the extraction chamber;

(7) For any residual non-polar analytes which may still be present in the hair sample, next change the temperature and pressure to optimize conditions for elution of any residual non-polar analytes still associated with the hair matrix, i.e., Condition #3, TABLE A or TABLE B, supra (EXAMPLE 13); and,

Collect the extracted presumptive polar and non-polar analytes in the extract flow for subsequent testing in an off-line testing procedure such as an immunoassay or GC/MS.

Illustrative Method #2:

Modifier and/or Additive in Equipment Packets

(1) Place hair sample and one or more equipment packet containing measured amounts of Modifier Stock Solution #1, 2 or #3 and/or Additive Stock Solution #1, #2 or #3 into the extraction chamber;

(2) After sealing the chamber, initiate flow of SF CO₂ and in using a short 3-5 minute static treatment mode bring both the temperature and pressure of the chamber to a temperature and pressure effective to (i) remove environmentally associated drug- and chemical-analytes while (ii) retaining metabolically incorporated drug analytes, e.g., a preferred or most preferred temperature and pressure as set forth in TABLE A and TABLE B, respectively, Condition #1, for non-polar analytes, and/or Condition #2, for polar analytes (EXAMPLE # 13, supra). However, selection of this temperature also being occasioned by (iii) the requirement that the equipment packet(s) is stable and not broken to release modifier and/or additive at the selected temperature and pressure;

(3) At the equilibrated temperature and pressure, initiate dynamic flow and wash the hair sample, i.e., at a flow rate according to Condition #1 or #2, supra, to remove the environmentally associated drug analyte(s) and chemical analyte(s) such as pesticides, herbicides, nicotine compounds and the like which may either constitute chemical analytes of interest, or alternatively, may constitute potential interfering or background substances in the detection assay;

(4) Collect the wash sample, or a portion of the wash sample, for an off-line testing procedure such as an immunoassay or GC/MS procedure;

(5) When the wash sample has been collected, terminate the flow of supercritical fluid and switch to a static extraction mode under the different conditions of temperature and/or pressure that are effective to both (i) break the equipment packet thereby releasing modifier and/or additive; and (ii) accomplish elution of any polar analytes incorporated into the hair matrix, e.g., a final in the chamber Condition #4 TABLE A or TABLE B, supra (EXAMPLE 13). For example, release of modifier and optional additive from the equipment packet may involve first increasing temperature, i.e., to break the packet, and then increasing pressure to the values set forth in TABLE A or TABLE B;

(6) Switch back to a dynamic mode of extraction by resuming flow of the supercritical fluid, i.e., at the flow rates indicated in either of TABLE A or TABLE B Condition #4, to elute the extracted polar analytes and then, as modifiers and any optional additives are progressively flushed from the extraction chamber;

(7) For any residual non-polar analytes which may still be present in the hair sample, next change the temperature and pressure to optimize conditions for elution of any residual non-polar analytes still associated with the hair matrix, i.e., Condition #3, TABLE A or TABLE B, supra (EXAMPLE 13); and,

(8) Collect the extracted presumptive polar and non-polar analytes in the extract flow for subsequent testing in an off-line testing procedure such as an immunoassay or GC/MS.

Illustrative Method #3:

In-Situ Derivatization: Modifier #4 and Additive #4, both in Equipment Packets

(1) Place hair sample and one or more equipment packet containing measured amounts of Modifier Stock Solution #4 and Additive Stock Solution #4 into the extraction chamber;

(2) After sealing the chamber, initiate flow of SF CO₂ and in using a short 3-5 minute static treatment mode bring both the temperature and pressure of the chamber to a temperature and pressure effective to (i) remove environmentally associated drug- and chemical-analytes while (ii) retaining metabolically incorporated drug analytes, e.g., a preferred or most preferred temperature and pressure as set forth in TABLE A and TABLE B, respectively, Condition #1, for non-polar analytes, and/or Condition #2, for polar analytes (EXAMPLE # 13, supra). However, selection of this temperature also being occasioned by (iii) the requirement that the equipment packet(s) is stable and not broken to release modifier and/or additive at the selected temperature and pressure;

(3) At the equilibrated temperature and pressure, initiate dynamic flow and wash the hair sample, i.e., at a flow rate according to Condition #1 or #2, supra, to remove the environmentally associated drug analyte(s) and chemical analyte(s) such as pesticides, herbicides, nicotine compounds and the like which may either constitute chemical analytes of interest, or alternatively, may constitute potential interfering or background substances in the detection assay;

(4) Collect the wash sample, or a portion of the wash sample, for an off-line testing procedure such as an immunoassay or GC/MS procedure;

(5) When the wash sample has been collected, terminate the flow of supercritical fluid and switch to a static extraction mode under the different conditions of temperature and/or pressure that are effective to both (i) break the equipment packet thereby releasing modifier and/or additive, i.e., the derivatizing agent; and (ii) accomplish derivatization and elution of any polar-derivatized analytes incorporated into the hair matrix, e.g., a final in the chamber Condition #3 TABLE A or TABLE B, supra (EXAMPLE 13) and/or the conditions of TABLE 3, EXAMPLE 6, supra. For example, release of modifier and optional additive from the equipment packet may involve first increasing temperature, i.e., to break the packet, and then increasing pressure to the values set forth in TABLE A or TABLE B;

(6) Switch back to a dynamic mode of extraction by resuming flow of the supercritical fluid, i.e., at the flow rates indicated in either of TABLE A or TABLE B Condition #3, to elute the extracted polar analytes and then, as modifiers and any optional additives are progressively flushed from the extraction chamber;

(7) For any residual non-polar analytes which may still be present in the hair sample, next change the temperature and pressure to optimize conditions for elution of any residual non-polar analytes still associated with the hair matrix, i.e., Condition #3, TABLE A or TABLE B, supra (EXAMPLE 13); and,

Collect the extracted presumptive polar and non-polar analytes in the extract flow for subsequent testing in an off-line testing procedure such as an immunoassay or GC/MS.

### EXAMPLE 15

### Equipment Packets

Temperature Sensitive Equipment Packet:

#1. Cellulose: Packets formed of cellulose acetate and cellulose nitrate are stable in organic solvents and in supercritical CO₂. However, including about 5% to about 20% by volume of a gas in the packet is commonly sufficient to insure that if temperature increases without a concomitant increase in pressure, the packet will break and release their constituent reagents.

#2. Poly-ethylene glycol: Polyethylene glycols (PEG) are wax polymers commercially available in several different molecular weights, e.g., PEG2000, PEG4000 and the like. PEG is relatively insoluble in organic solvents, except at elevated temperature where melting of the polymer permits miscibility in organic solvents.

Temperature/Pressure Sensitive Equipment Packet:

#1: Lactose polymers: Lactose polymers are stable in organic solvents, except under conditions of elevated pressure and temperature where denaturation weakens the cross-links and leakage and ultimate breakage occurs.

#2. Dextrin polymers: Like lactose, dextrin polymers are stable in organic solvents, except under conditions of elevated pressure and temperature and pressure where denaturation forces breakage.

#3. Plastic polymers: Plastic copolymers are available having different melting points and stability in organic solvents. Melting at higher temperatures causes release of constituent modifiers and/or additives.

### EXAMPLE 16

### Trapping to Collect Extracted analytes

Extract Trapping: A variety of different traps may be employed to collect the instant derivatized and non-derivatized drug and chemical analytes from SFE fluids. For example, collection may be off-line, i.e., through a restrictor allowing controlled decompression into a liquid such as water or an organic solvent; or, onto the surface of an inert solid phase or through a solid phase such as a filter; or, onto a surface such as glass beads or a polymer such as may subsequently be useful in performing an immunoassay (e.g., as set forth supra). Controlled decompression with simultaneous pressurized solvent trapping is also envisaged, i.e., by pressurizing a solvent stream into the decompressing supercritical extract. Alternatively, in-line collection methods may also be employed including collection of the instant derivatized and non-derivatized drug and chemical analytes from SFE fluids into column matrices such as commonly used in GC/MS, e.g. SPE cartridges and the like. Collection is preferably accomplished by adjusting the temperature and pressure of the restrictor, preferably, to about 40° C. to about 50° C., and any extract collection vials, preferably, to about 4° C. to about 10° C., so as to minimize clogging of the restrictor, as well as, decreasing potential evaporative and aerosol losses analytes. In situations where solid phase trapping is advantageous, analytes may subsequently be eluted from the trap for use in analytical assays such as immunoassays and GC/MS. Representative examples of the subject solid phase traps include silica, cellulose, anion and cation exchange resins, octyldecylsilane (ODS), Tenax, Porapak-Q, silica bonded with diol, florisil, basic and neutral alumina, charcoal and the like. Preferably, the subject solid phase traps are contained within about a 1 ml volume collector cartridge. Representative examples of solvents that may be used to elute analytes from solid phase traps include acetone, methanol, ethyl acetate, acetonitrile and the like. Elution is not absolutely required since it is envisaged that certain analytical assays, e.g., immunoassays, may be conducted either in-line through the trap; or alternatively, on one or more surfaces within the trap; or, on solid phase samples removed from the trap for analysis. In one presently preferred embodiment a trap housing is a clear plastic polymer or glass collector cartridge and the subject analytical assay is conducted by visually inspecting the cartridge for the presence of a detectable signal product from a signal generating compound housed within the trap solid phase material. For purposes of protecting the subject glass or plastic against temperature and pressure, it may optionally be contained within a stainless steel sleeve housing.

### EXAMPLE 17

### Detection of Steroids in Hair Samples Collected from Domestic Animals

Steroids are present in hair samples as unconjugated phenolic steroids, glucuronide conjugated steroids and mono- and disulfate conjugated steroids. Each of the latter respective conjugates is prepared for SFE as follows: namely,

Glucuronide steroids in hair are liberated by pre-treating with β-glucuronidase (Sigma Fine Chemicals, St. Louis, Mo.) at 37° C. for 1-24 hrs.

Steroid sulfate conjugates are liberated by solvolysis in acidified tetrahydrofuran as described in EXAMPLE 9, above.

Prior to SFE, steroid hydroxyl groups are preferably derivatized according to EXAMPLE 7) above. As an alternative detection method, steroid hydroxyls may be derivatized to steroidal thiophosphinic esters for phosphorous detection, e.g., similar to derivatization methods utilized by David and Novotny (HAIRSFE-308) for phosphorus thermionic detection of steroids in urine and plasma. Phosphorous is, alternatively, detected colorimetrically using e.g. a commercially available Alkaline phosphatase immunoassay substrate. SFE conditions for isolating steroids and their conjugates from hair are conducted in nitrous oxide at a temperature of about 70° C. to about 100° C. and about 240 atm to about 285 atm. Elution of the subject derivatized steroids is accomplished in about 10 minutes to about 30 minutes. Where there is interest in separating unconjugated (non-metabolized) steroids from conjugated, this is accomplished by collection of samples at different time points during elution: i.e., unconjugated steroids elute first followed by glucuronidated and sulfated steroids, respectively. Androstrone, pregnanediol, estrogens and pregnanes (C₂₁ steroids) may be used as reference standards for purposes of calibration, control and determining specificity.

### EXAMPLE 18

### Detection of Pesticides, Steroids and Chemical Analytes in Hair Samples Collected from Man and Domestic Animals

Use of anabolic steroids to enhance performance in sports is wide spread. Illegal use of steroids to enhance weight gain in domestic animals is also reportedly wide spread. Antimicrobial agents are also commonly used to enhance weight gain, and their use is now discouraged because of the medical consequences for humans associated with emergence of drug resistance microorganisms. Water gain and decreased fat content induced by β-agonists, e.g. clenbuterol, has also led to agricultural and sports abuses. Contamination of food products with pesticide residues is also becoming of increasing concern. Hair is a convenient sample in testing to alleviate these respective different concerns.

Steroids: Methyltestosterone, nortestosterone and/or testosterone is detectable in the hair of man and domestic animals. Unlike urine, hair does not commonly contain endogenous steroids or dietary components that interfer with detection of target analytes. Steroids such as nortestosterone and testosterone and their glucuronide metabolites have only limited solubility in supercritical CO₂. Extraction from hair is accomplished in CO₂ modified with MeOH and H₂O. Glycoconjugate-matrix treatment.

Analgesic and Non-Steroidal Anti-Inflammatory Agents: In intensive care and emergency medicine it is often desirable to determine whether a subject has had relatively long-term exposure of drugs which are not drugs of abuse. For instance, liver damage can result from overuse or misuse of analgesic agents and nonsteroidal anti-inflammatory agents. The subject drug analytes may be detected in hair samples.

Antimicrobial Agents: Antimicrobial drug analytes can be used to achieve unlawful weight gain in livestock. The subject drug analytes may be detected in hair. Representative antimicrobial drug analytes include sulfonamides such as sulfamethazine, sulfadimethoxine, sulfaquinoxaline, sulfamethoxazole and the like.

β-Agonists: β-Agonists have been used in agriculture to achieve rapid weight gain in livestock. According to the instant methods, β-Agonists present in hair are extractable using supercritical fluid CO₂ modified with methanol (10-20% v/v in CO₂) and containing TEA or DEA and/or TFAA as additives, i.e., each of which additives is present at 0.1-0.5% v/v in the methanol.

Pesticides and Herbicides: Environmental exposure of animals to pesticides and herbicides is determined according to the instant methods by testing unwashed animal hair, e.g., using SF CO₂.

### EXAMPLE 19

### Detection of Glycolipids in Hair Samples Collected from Domestic Animals

With the recent European epidemic of bovine spongiform encephalitis (BSE) and the possible presence of scrapie and other prion agents in sheep brain, there is growing concern about practices of feeding animal meals containing sheep brain parts. Environmental exposure of other species to animal meals including sheep brain may be detected by extracting the hair, e.g., SFE, and testing for the presence of sheep brain glycosphingolipid (glycolipid) antigens in hair. Glycolipids are composed of a sugar chain linked together and to sphingosine through glycosyl bonds. The sugar chain confers antigenicity to the molecule and such antigens are also known in the art as blood group antigens. In vitro, glycolipids in micellar or liposomal forms are incorporated into cellular lipid membranes. In vivo, environmental glycolipids are incorporated into skin, hair follicles and hair. Recent environmental exposure is determined by testing for glycolipid passively associated with hair, e.g., using supercritical CO₂ washing. Longer-term environmental exposure is determined by extracting matrix bound glycolipid, e.g., according to the EXAMPLES disclosed supra. It is anticipated that wet chemical methods, e.g. solvent washing, treating with acid and/or base and/or enzymatic digestion, will also be usefuil in liberating environmentally associated glycolipid and matrix entrapped glycolipid. Gangliosides, a sialic acid containing class of glycolipids, are the presently preferred glycolipids indicative of exposure to brain. Ganglioside antigens are present in large amounts in brain, in smaller amounts in other tissues and are virtually absent in normal hair. Detection of ganglioside antigens in hair is accomplished using immunoassays (supra) and/or chemical detection, e.g., chemical detection of sialic acid. Representative sheep brain gangliosides for detection include GM3, GM2, GM1, GD1a, GD1b and the like.

### EXAMPLE 20

### Compositions and Methods for Producing Controls

As set forth supra, extraction of drug metabolites from the matrix of drug user hair is not mirrored by either of spiked hair samples, i.e., drug added to normal hair prior to the assay, or drug fortified hair, i.e., hair soaked in drug and DMSO. Development and manufacturing of commercial assays requires quality controls, i.e., for assurance (QA) and control of possible lot-to-lot variation (QC). Extraction and detection of drug analytes also requires in-assay positive and negative controls, as well as, possible calibrators. Shipping drugs of abuse as assay standards is problematic. It was considered highly desirable to be able to produce, at will, samples of animal hair that would mimic human drug-user hair. Oral or intravenous dosing of animals with drugs of abuse was also considered problematic.

Embodiments of the invention provide liposome formulations containing one or more drugs of abuse, and methods for topical treatment of animals to effect incorporation of the subject drugs into hair follicles and thence, metabolically, the drugs are incorporated into hair. Representative liposomes useful according to the invention include unilaminar and multilaminar liposomes constructed using phosphatidyl choline (PC), phosphatidyl ethanolamine (PE), phosphatidyl serine (PS), glycolipids and the like. Animals, e.g., sheep, are subject to repeated topical dosing at the same local site until levels of drug in the subject animal hair approach those in drug user hair, or approach the levels desired in specific assay calibrators.

### Materials and Methods

Chemicals and Reagents. All reagents are commercially available and are preferably Reagent Grade, HPLC grade, GC/MS grade or better.

Target Analvtes. Target drug analytes as set forth in TABLE 1 are commercially available.

CO₂: CO₂ gas provided by the specialty gas industry is potentially contaminated with low levels of impurities including lubricants increasing the background in GC/MS assays. High purity GC-grade CO₂ is used and where necessary CO₂ is purified using in line catalyst-based purification systems such as that disclosed by Zorn et al. (SFE-126). Supercritical sources of CO₂ may utilize CO₂ pumped to achieve the desired density and/or CO₂ pressurized in a tank with a helium headspace.

1. A method for testing to determine the presence or amount of an analyte in a biological sample collected from a subject, comprising the steps of:
simultaneous on-line extraction of the analyte from the sample into a supercritical fluid by,
collecting the sample from the subject and placing the sample into an extraction chamber;
exposing the sample in the extraction chamber to a supercritical fluid to extract the analyte from the sample;
eluting the extracted analyte from the extraction chamber;
collecting the eluate containing the extracted analyte;
testing the eluate to determine the presence or amount of the analyte; and,
determining that if the analyte is present said analyte was present in the sample.

2. A supercritical fluid extraction method for screening a subject to determine the possible presence of a drug analyte, wherein the drug usage by said subject is unknown, comprising the steps of collecting a biological sample from the subject; simultaneously extracting any analyte present in the sample by first treating the sample with a derivatizing agent effective to neutralize the chemical charge of one or more substituent charged groups in said analyte and thereafter extracting any derivatized drug analytes in said sample into a supercritical fluid; and, collecting said supercritical fluid and testing to detect the presence or amount of said derivatized analyte.

3. The method of claim 1 or 2, wherein the supercritical fluid is selected from group consisting of carbon dioxide, nitrous oxide, pentane and butane.

4. The method of claim 2, wherein said analyte substituent groups are selected from the group consisting of hydroxyl, carboxyl, amide, amine and thiol.

5. The method of claim 2, wherein said derivatizing agent is selected from the group consisting of an alkylating reagent, an acylating reagent and a silylating reagent.

6. The method of claim 3, wherein said supercritical fluid comprises CO₂.

7. The method of claim 6, wherein said supercritical fluid additionally comprises CO₂ about 1% (v/v) to about 30% (v/v) of a modifier solution.

8. The method of claim 7, wherein said modifier comprises an organic solvent selected from the group consisting of methanol, dichloromethane , ethanol, ethyl acetate, isopropanol, and hexane.

9. The method of claim 7, wherein said CO₂ supercritical fluid additionally comprises about 0.5% (v/v) to about 1.2% (v/v) of an additive solution and/or water.

10. The method of claim 9, wherein said additive solution comprises an amine additive selected from the group consisting of methylamine, dimethylamine, trimethylamine and butylamine and/or water.

11. The method of claim 5,. wherein said supercritical fluid additionally comprises an alkylation catalyst.

12. The method of claim 1, wherein said analyte is selected from the group consisting of a drug of abuse, a pesticide, an herbicide, an environmental toxin and a carcinogen.

13. The method of claim 12, wherein said analyte is a drug of abuse selected from the group consisting of cocaine, heroin, morphine, 6-monoacetylmorphine, codeine, opium, methamphetamine, amphetamine, MDA, MDMA, PCP, Δ-9-THC, Δ-9-THC-acid, phencyclidine, and metabolites thereof.

14. The method of claim 1 or 2, wherein said subject is a human or a domestic animal.

15. The method of claim 1 or 2 comprising an additional step of washing with supercritical CO₂ to remove possible environmental contamination before said testing step.

16. The method of claim 1 or 2, wherein said detection step is selected from group consisting of an immunoassay test and a GC/MS test.

17. A method for a supercritical fluid extraction of an analyte having a reactive hydrogen group from a biological sample, comprising the step of treating the sample with a derivatizing agent prior to initiating the supercritical fluid extraction.

18. The method of claim 17, wherein said reactive hydrogen group is selected from the group consisting of an alcohol, an amide, an amine, an aryl and a thiol.

19. The method of claim 18, wherein said derivatizing agent is selected from the group consisting of an acylating agent, an esterification agent, an alkylating agent, a methylating agent and a silylating agent.

20. The method of claim 19, wherein said acylating reagent is selected from the group consisting of an anhydride, an acyl halide, an activated acyl amide.

21. The method of claim 20, wherein said activated acyl amide comprises acylimidazole or bis(acylamide).

22. The method of claim 20, wherein said acyl halide comprises a halogenated acylation reagent selected from the group consisting of a perfluoro acid anhydride, a perfluoroimidazole and an N-methyl-bis[trifluoroacetamide].

23. The method of claim 22, wherein said perfluoro acid anhydride is selected form the group consisting of trifluoroacetic acid anhydride (TFAA), pentafluoropropionic acid anhydride (PFPA) and heptafluorobutyric acid anhydride (HFBA).

24. The method of claim 22, wherein said perfluoroimidazole is selected from the group consisting of trifluoroacetylimidazole (TFAI), pentafluoropropionylimidazole (PFPI) and heptafluorobytyrylimidazole (HFBI).

25. The method of claim 22, wherein said N-methyl-bis[trifluoroacetamide] comprises N-methyl-bis-(trifluoroacetamide) (MBTFA).

26. The method of claim 17, wherein said analyte is a drug analyte selected from the group consisting of methamphetamine, amphetamine, methylenedioxyamphetamine (MDA), methylenedioxymethamphetamine (MDMA), and methylenedioxyethylamphetamine (MDEA).

27. A method for a supercritical fluid extraction of an analyte having a reactive carboxyl group from a biologocial sample, comprising the step of treating the sample with a derivatizing agent prior to initiating the supercritical fluid extraction.

28. The method of claim 27, wherein said derivatizing agent comprises an esterification reagent.

29. The method of claim 28, wherein said esterification reagent is selected from the group consisting of a pentafluoropropanol and an anhydride.

30. A method for a supercritical fluid simultaneous extraction of two or more analytes from biological sample, wherein said analytes comprise both a reactive hydrogen group and a reactive carboxyl group, comprising the step of treating the sample with a derivatizing agent effective to derivatize both the reactive hydrogen and the reactive carboxyl.

31. The method of claim 30, wherein said derivatization agent comprises a perfluoro acid anhydride.

32. The method of claim 31, wherein said reactive hydrogen is acetylated and said reactive carboxyl is esterified.

33. The method of claim 32, wherein said esterification of the carboxylic acid groups with the acid anhydride result in the formation of a pentafluoropropyl-ester-drug analyte-derivative.

34. The method of claim 30, wherein said analyte is a drug analyte selected from the group consisting of methamphetimine, amphetamine, MDMA, MDA, cocaine, benzyoylecgonine, cocaethylene, heroin, morphine, codeine, 6-MAM, delta-9-THC, delta-9-THC-9-carboxylic acid, dihydroxyl THC, PCP, Nicotine and cotinine.

35. A method for a supercritical fluid simultaneous extraction of two or more analytes from a biological sample, wherein said analytes comprise both a reactive hydrogen group and a reactive carboxyl group, comprising the step of treating the sample with two or more derivatizing agents effective to derivatize both the reactive hydrogen and the reactive carboxyl.

36. The method of claim 35, wherein said two or more derivatizing agents are added simultaneous or sequentially to an supercritical extraction fluid.

37. The method of claim 36, comprising addition of a perfluoro-anhydride and a perfluoro-alcohol to the supercritical extraction fluid.

38. The method of claim 17, comprising the additional step of adding a modifier to said supercritical fluid.

39. The method of claim 38, wherein said modifier comprises an organic solvent selected from the group consisting of dichloromethane, dichloroethane, methanol, acetone, ethanol, ethyl acetate and methyl acetate.

40. The method of claim 17, comprising the additional step of adding an additive to said supercritical fluid.

41. The method of claim 40, wherein said additive comprises an organic amine selected from the group consisting of diethylamine, triethylamine and butylamine.

42. The method of claim 40, wherein said additive comprises an organic buffer and a polyethylene-glycol.

43. The method of claim 40, wherein said organic buffer comprises about 0.1 mM to about 10 mM Tris base.

44. The method of claim 40, wherein said polyethylene glycol comprises about 1% (v/v) to about 15% (v/v) of polyethylene glycol having a polymer molecular weight of about 1500 to about 3000.

45. The method of claim 40, wherein said additive comprises about 0.1% (v/v) to about 5% (v/v) of a zwitterionic compound.

46. The method of claim 45, wherein said zwitterionic compound is selected from the group consisting of a zwitterionic detergent, an amino acid, a glycolipid and an amino sugar.

47. The method of claim 46, wherein said zwitterionic detergent is selected from the group consisting of 3-[(3-chloramidopropyl)dimethylammonio]-2-hydroxy-1-propanesulfonate (CHAPSO), 3-[(3-chloramidopropyl)dimethylammonio]-1-propanesulfonate (CHAPS), N-decyl-N,N-dimethyl-3-ammonio-1-propane-sulfonate (NDAPS) and phosphatidyl-choline dipalmitoyl.

48. The method of claim 47, wherein said zwitterionic detergent additive further comprises about 1% (v/v) to about 20% (v/v) of an antifoaming agent.

49. The method of claim 48, wherein said antifoaming agent is selected from the group consisting of a polyol, a mixture of a silicone and a polyols and a silicone.

50. The method of claim 49, wherein said polyol comprises Antifoam 204.

51. The method of claim 49, wherein said mixture of silicones and polyols comprises Antifoam 289.

52. The method of claim 49, wherein said silicone comprises Antifoam A or Antifoam B.

53. The method of claim 38, wherein said additive comprises an anionic detergent, a cationic detergent and antifoaming agent.

54. The method of claim 53, wherein said anionic detergent is selected from the group consisting of an alginic acid, a caprylic acid, a cholic acid, a decane sulfonic acid, a dehydrocholic acid, a deoxycholic acid, a dioctyl sulfosuccinate, a dodecanesulfonic acid, a glycocholic acid, a glycodeoxycholic acid, a hepatane sulfonic acid, a hexane sulfonic acid, a lauroylsarcosine, a lauryl sulfate, a nonanesulfonic acid, an octanesulfonic acid, a pentanesulfonic acid, a taurocholic acid, a taurodeoxycholic acid, a Teepol HB7, a Tergiton and a Triton.

55. The method of claim 54, wherein said alginic acid comprises a β_{1,4}-D-mannuronic acid polymer.

56. The method of claim 53, wherein said cationic detergent is selected from the group consisting of an alkyltrimethylammonium bromide, a benzalkonium chloride, a benzyldimethyldodecyl ammonium bromide, a benzyldimethylhexadecyl ammonium chloride, a benzyldimethyltetradecyl ammonium chloride, a cetyldimethylethyl ammonium bromide, a cetylpyridinium bromide, a cetylpyridinium chloride, a decamethonium bromide, a dimethyldioctadecyl ammonium bromide, a methylbenzethonium chloride, a methyl mixed trialkyl ammonium chloride, a methyl trioctylammonium chloride, and an N,N′,N′-polyoxyethylene(10)-N-tallow-1,3-diamino propane.

57. The method of claim 53, wherein said antifoaming agent is selected from the group consisting of a polyol, a mixture of a silicone and a polyol and a silicone.

58. The method of claim 57, wherein said polyol comprises Antifoam 204.

59. The method of claim 57, wherein said mixture of a silicone and a polyol comprises Antifoam 289.

60. The method of claim 57, wherein said silicone comprises Antifoam A or Antifoam B.

61. A method for testing a biological sample to distinguish between environmental exposure to an analyte and metabolic incorporation of the analyte, comprising the steps of:
(i) placing said sample and an equipment packet comprising a modifier and an optional additive into an extraction chamber suitable for maintenance of both a static mode and a dynamic flow mode of a supercritical fluid;
(ii) initiating a dynamic flow mode effective to washing said sample in said extract chamber with a supercritical fluid under physical conditions of temperature and pressure that are effective to remove analyte that is environmentally associated but not analyte that is metabolically incorporated into said sample and wherein said conditions are not effective to release the modifier or additive from the equipment packet;
(iii) collecting a wash sample for an off-line testing procedure and terminating said flow of supercritical fluid to switching from said dynamic mode to a static mode;
(iv) in a static mode, changing said physical conditions to conditions of temperature and pressure effective to release said modifier and optional additive from said equipment packet and maintaining said static mode for a period of time effective to extract the metabolically incorporated analyte;
(v) switching to a dynamic mode by resuming flow of said supercritical fluid; and changing said physical conditions to conditions of temperature and pressure effective to extract the metabolically incorporated analyte;
(vi) collecting an extract sample for an off-line testing procedure;
(vii) testing said wash sample to determine the presence or amount of said environmentally associated analyte;
(viii) testing said extract sample to determine the presence or amount of said metabolically incorporated analyte;
wherein, a positive test result in step (vii) and a negative test result in step (viii) is effective to establish said environmental exposure to said analyte without metabolic incorporation; a negative test result in step (vii) and a positive test result in step (viii) is effective to establish metabolic incorporation of said analyte without environmental exposure; and, a positive test result in both step (vii) and step (viii) is effective to distinguish both environmental exposure and metabolic incorporation of said analyte into said sample.

62. A method for extracting an analyte from a biological sample comprising the step of incubating the sample with an enzyme capable of degrading a matrix glycoconjugate of the sample for a period of time and under conditions effective to hydrolyze said glycoconjugate.

63. The method of claim 62, wherein said enzyme is selected from the group consisting of a hexosaminidase, an endoglycosidase, a sialidase, a galactoaminidase, a glucosaminidase, a glucosidase, a galactosidase, a phytase and a chitinase.

64. A supercritical fluid extraction equipment packet comprising a solution selected from the group consisting of a modifier stock solution, an additive stock solution and a derivatizing agent solution.

65. The equipment packet of claim 64, wherein said modifier stock solution comprises an organic solvent selected from the group consisting of dichloromethane, dichloroethane, methanol, acetone, ethanol and ethyl acetate.

66. The equipment packet of claim 65, wherein said additive stock solution comprises, comprising one or more detergents selected from the group consisting of a zwitterionic detergent, an anionic detergent and a cationic detergent.

67. A supercritical fluid extraction method for screening a subject to determine the possible exposure to, or ingestion of, or inhalation of an animal brain product, comprising the steps of collecting a biological sample from the subject, extracting complex carbohydrates from the sample and testing to determine the presence or amount of an animal brain complex carbohydrates present in said sample.

68. The method of claim 67, wherein said complex carbohydrates are selected from the group comprising glycoprotein and glycolipid blood group antigens.

69. The method of claim 67, wherein said complex carbohydrate comprises a glycolipid selected from the group consisting of a neutral glycolipid, a ganglioside and a sulfatide.

70. A method for simultaneously extracting an analyte from a biological sample having glycoconjugate matrix comprising the steps of treating the sample with a derivatizing agent effective to neutralize the chemical charge of one or more sugar ring substituent groups in said glycoconjugate matrix; and, extracting the analytes in a supercritical fluid.

71. A supercritical fluid extraction method for extracting an analyte from a biological sample, comprising the step of bringing the sample into contact with a modifier solution comprising an anionic detergent, a cationic detergent and an organic solvent, for a period of time and under conditions effective to extract said analyte from said sample.

72. The method of claim 1, 2, 17, 27, 30, 35, 61, 62, 67, 70 or 71, wherein the biological sample is selected from the group consisting of hair, feathers, nails, hoofs, skin and muscle.

73. The method of claim 1, 2, 17, 27, 30, 35, 61, 62, 67, 70 or 71, wherein the analyte is selected from the group consisting of a polar, a non-polar analyte and a combination of both.

74. The method of claim 1, further comprising exposing the sample to a derivatizing agent in an amount effective to form derivatives of the analyte.

75. The method of claim 74, wherein the analyte is a polar analyte.

76. The method of claims 1, 2, 17, 27, 30, 35, 61, 62, 67, 70 or 71, wherein the analyte is selected from the group consisting of a drug analyte and a chemical analyte.
